(19) United States
(12) Patent Application Publication
(10) Pub. No.: US 2021/0260312 A1
(43) Pub. Date: Aug. 26, 2021

(54) ELECTROMECHANICAL APPARATUS AND METHOD FOR USING A MOBILE INHALER

(71) Applicant: NEXVAP SA, Puidoux (CH)

(72) Inventors: Julien Lacour-Gayet, Lacroix sur Lutry (CH); Philippe Lacour-Gayet, Bel Ombre (MU); Francesco Guidetti, Chernex (CH); Giorgio Leuzzi, Roma (IT); Vicenzo Stornelli, Roio Poggio (IT); Leonardo Pantoli, Coppito (IT); Gianluca Barile, Avezzano (IT); Mirco Muttillo, Baranello (IT); Alfiero Leoni, Alfedena (IT); Stefano Ricci, L'Aquila (IT); Andrea Pelliccione, Coppito (IT); Walter D'Ambrogio, Frosinone (IT); Michele Gabrio Ernesto Antonelli, Termoli (IT); Jacopo Brunetti, L'aquila (IT)

(21) Appl. No.: 17/252,249

(22) PCT Filed: Jun. 14, 2019
(86) PCT No.: PCT/IB2019/000819

§ 371 Date: Dec. 14, 2020

Related U.S. Application Data
(60) Provisional application No. 62/685,083, filed on Jun. 14, 2018.
(60) Provisional application No. 62/690,630, filed on Jun. 27, 2018.

(51) Int. Cl. A61M 15/06; A61M 15/00; B05B 17/06

(52) U.S. Cl. CPC A61M 15/06; A61M 2207/00; A61M 15/0085; A61M 15/002; A61M 15/0013; A61M 15/0011; B05B 17/0607; A61M 2205/8206; A61M 2205/609; A61M 2205/3584; A61M 2205/583; A61M 2205/505; A61M 2205/52; A61M 2205/3334; A61M 2209/088; A61M 15/0021

(57) ABSTRACT

A mobile inhaler is configured to provide an inhalable aerosol to a user. The mobile inhaler may include a vibrating mesh atomizer. The vibrating mesh atomizer may include a vibrating mesh membrane comprising a plurality of holes, a piezoelectric actuator coupled to the vibrating mesh membrane, wherein the processing equipment is configured to actuate the piezoelectric actuator, and a vibrating mesh membrane holder. The mobile inhaler includes processing equipment, which may include memory configured to store information about one or more users, including usage behaviour. A capsule is used to hold liquid for atomizing and may include a plunger to dispense liquid. The processing equipment may be configured to determine desired dosages, or other desired usage metrics, based on the stored information. In some embodiments, information from a plurality of mobile inhalers may be stored and analysed.

### CROSS-REFERENCE TO RELATED APPLICATIONS

The present disclosure is directed towards mobile inhalers, and more particularly towards mobile inhalers for delivering aerosol. This application claims the benefit of U.S. Provisional Patent Application No. 62/685,083 filed Jun. 14, 2018, and U.S. Provisional Patent Application No. 62/690,630 filed Jun. 27, 2018, the disclosures of which are all hereby incorporated by reference herein in their entireties.

### BACKGROUND

An inhaler is an apparatus used for delivering compounds to the body via the lungs. Several types of inhalers are known in the art, for example the dry powder inhaler, pressurized metered-dose inhaler (MDI), and the nebulizer.

More specifically, an inhaler is a medical apparatus used for delivering a substance (e.g., medication) into the body via the lungs. Several types of inhalers are known in the art. The most common type of inhaler is the pressurized metered-dose inhaler (MDI). In MDIs, medication is typically stored in solution in a pressurized canister that contains a propellant, although it may also be a suspension. The MDI canister is attached to a plastic, hand-operated actuator. On activation, the MDI releases a fixed dose of medication in aerosol form. The aerosolized medication is drawn into the lungs by a user continuing to inhale deeply before holding their breath for a few seconds to allow the aerosol to settle onto the airways of the lungs. Another type of inhaler is the dry powder inhaler that releases a metered or apparatus-measured dose of powdered medication that is inhaled through the apparatus. A nebulizer is a further type of inhaler that typically supplies the medication as an aerosol created from an aqueous formulation.

In the last decade, a new way of nicotine delivery to the lung has started to gain popularity, the so-called e-cigarettes or Electronic Nicotine Delivery System (ENDS).

In an e-cigarette, or ENDS in general, a liquid composition containing nicotine, propylene glycol, vegetable glycerin and flavorings, is evaporated by means of heating a coil surrounding a wick. The wick is a fabric, usually cotton, through which liquid is drawn by capillarity. When the coil, being an electrical resistance, surrounding the wick is connected to power source, it heats the liquid to temperatures of up to about 300° C., thereby causing the vaporization of the liquid contained in the wick, and the user then inhales this vapor. Although an e-cigarette or ENDS has recently been recognized as being a safer alternative to a tobacco cigarette (e.g. Public Health England recognizing that e-cigarettes are 95% less risky than combustible tobacco), there are still different disadvantages linked with the use of e-cigarettes. Because the liquid composition is being vaporized, traces of toxic or potentially toxic compounds might still be produced when an e-cigarette is used, through various heat-induced chemical reactions. The vapor can contain toxicants and traces of heavy metals (at levels permissible in inhalation medicines), and potentially harmful chemicals not found in tobacco smoke. To a lesser extent, some materials used in e-cigarettes (e.g., metals and plastics) may generate additional harmful chemical compounds when the temperature is elevated.

Current inhalers are difficult to hand carry and must be kept in a vertical or upright position when nebulizing, as well as to prevent the leakage or spillage of the liquid from the tank or capsule where it is stored. Current inhalers generally do not evaluate or meter dose precisely the quantity of aerosol generated at any given time interval by the patient or consumer.

### SUMMARY

In some embodiments, the present disclosure provides a novel mobile inhaler that allows a user to inhale nicotine, among other substances, while at the same time overcoming the various problems associated with using e-cigarettes.”

Some embodiments of the present disclosure provide a nicotine delivery system that requires no heating to generate nicotine contacting nanoparticles that are delivered to the deep lung. This non-heating delivery mechanism provides the safer generation nanoparticle generation since there is no heat-induced structural changes to the chemical compounds being aerosolized. The delivery of nicotine to the deep lung also increased nicotine delivery efficiency and thereby provides for a more satisfying nicotine experience by the user with less concentration of nicotine, thereby providing a potential for less addictive nicotine delivery product. Furthermore, the inclusion of metered-dosage and communication electronics provides the basis of a successful nicotine replacement therapy.

In some embodiments, the present disclosure relates to an apparatus, compound containing liquids and a method for delivering liquid compounds, using vibrating mesh technology, including a novel electronic circuity and architecture.

In some circumstances, it has been recently recognized that electronic nicotine delivery systems (ENDS) are a safer alternative to combustible cigarettes, however, continued developments are needed to more effectively and accurately deliver compounds, in a risk-free process, via aerosol delivery. More specifically, technically advanced circuits and architectures have the potential to bring the ENDS technology to higher level of efficiency and consumer acceptability. The improved ability to deliver compounds through innovative aerosol delivery carriers is important to advance the use and acceptance of ENDS for both recreational and medical use. For these reasons the disclosed apparatus and method for delivering compounds, using advanced and novel circuity and architecture for aerosol inhalators overcomes the drawbacks of the currently available apparatuses and methods.

The apparatus and method are based on the use of a micro-pump that transfers liquid in measured quantities from a hermetic liquid container or capsule to a mesh-type nebulizer, which in turn generates an aerosol with droplets of controlled size that suitable for deep lung penetration.

In some embodiments, the apparatus and method include a novel electronic circuitry that controls the generation of either variable or controllable aerosol volumes. The size and shape of the apparatus is such, that it can be easily held with one hand and is usable in all positions held. The apparatus is rechargeable, both in terms of electric energy and refillable in terms of liquid to be nebulized.

In some embodiments, the present disclosure further relates to an apparatus that functions as a mobile inhaler for delivering liquid compositions that contain compounds for both recreational and/or medicinal use. More specifically, the liquid compositions are designed to, deliver soluble or miscible chemical compounds.

In some embodiments, the present disclosure relates to a mobile inhaler or electronic cigarette delivering liquids or liquid compounds in the form of aerosol, using vibrating mesh technology and an electric motor, controlled by an electronic board. In some embodiments, for example, the electronic board includes a dedicated motor controller.

In some embodiments, the present disclosure is directed to a mobile inhaler. The mobile inhaler includes a mouthpiece having one or more air ducts configured for intaking air and an aperture configured to interface to a user and deliver aerosol to the user during inhalation. The mobile inhaler also includes a body coupled to the mouthpiece, and a replaceable capsule configured to contain a liquid, and removably mounted in the body. The mobile inhaler also includes a power source configured to provide electrical power, processing equipment coupled to the power source, a liquid pump system configured for pumping the liquid, and a vibrating mesh atomizer configured for atomizing the liquid. The liquid pump system is further configured to pump the liquid to the atomizer.

In some embodiments, the present disclosure is directed to a mobile inhaler configured to atomize a liquid for inhalation. The mobile inhaler includes processing equipment, a liquid pump system coupled to the processing equipment and configured for pumping the liquid. and a vibrating mesh atomizer configured for atomizing the liquid. The liquid pump system is further configured to pump the liquid to the atomizer. The vibrating mesh atomizer includes a vibrating mesh membrane having a plurality of holes, a piezoelectric actuator coupled to the vibrating mesh membrane, and a vibrating mesh membrane holder configured to house the vibrating mesh membrane and a liquid membrane chamber configured to hold the liquid in contact with the vibrating mesh membrane. The processing equipment is configured to actuate the piezoelectric actuator.

In some embodiments, the present disclosure is directed to a mobile inhaler. The mobile inhaler includes a mouthpiece, a body coupled to the mouthpiece, a replaceable capsule configured to contain a liquid and removably mounted in the body, a power source configured to provide electrical power, processing equipment coupled to the power source, a liquid pump system configured for pumping the liquid, and a vibrating mesh atomizer configured for atomizing the liquid. The processing equipment includes memory configured to store information about the user. The liquid pump system is further configured to pump the liquid to the atomizer.

In some embodiments, the present disclosure is directed to a mobile inhaler system configured to atomize a liquid for inhalation by a user. The mobile inhaler system includes processing equipment that includes memory configured to store information about the user, and a communications interface. The mobile inhaler also includes a liquid pump system configured for pumping the liquid, and a vibrating mesh atomizer configured for atomizing the liquid. The liquid pump system is further configured to pump the liquid to the atomizer. In some embodiments, the system includes an external device communicatively coupled to the communications interface by a communications link.

### BRIEF DESCRIPTION OF THE DRAWINGS

The present disclosure is further described in the various embodiments, which are illustrated my means of the following drawings, wherein:

FIG. 1 shows a cross-sectional view of the apparatus with capsule inserted, in accordance with some embodiments of the present disclosure;

FIG. 2 shows a cross-sectional view and exploded view (e.g., of the apparatus of FIG. 1) of micro-pump and needle, membrane chamber, piezoelectric ring, membrane, and membrane fixer blocks, in accordance with some embodiments of the present disclosure;

FIG. 3 shows a general structure of an illustrative vibrating mesh membrane holder, in accordance with some embodiments of the present disclosure;

FIG. 4 shows a cross-sectional view of an illustrative vibrating mesh membrane holder, in accordance with some embodiments of the present disclosure;

FIG. 5 shows a cross-sectional view of an illustrative vibrating mesh membrane holder, in accordance with some embodiments of the present disclosure;

FIG. 6 a cross-sectional view of an illustrative vibrating mesh membrane holder, in accordance with some embodiments of the present disclosure;

FIG. 7 shows an elevation perspective view of the apparatus of FIGS. 1-6, in accordance with some embodiments of the present disclosure;

FIG. 8: Cross-section of the liquid container or capsule, in accordance with some embodiments of the present disclosure;

FIG. 9: Plan of the electronic board or CPU, and general operating scheme of the electronics of the apparatus, in accordance with some embodiments of the present disclosure;

FIG. 10: Plan of the power converting unit optionally including a DC/AC converter, in accordance with some embodiments of the present disclosure;

FIG. 11: Operating modes (a), (b) and (c) of the electronic board or CPU, in accordance with some embodiments of the present disclosure;

FIG. 12: Operating modes (d), (e), (f) of the electronic board or CPU, in accordance with some embodiments of the present disclosure;

FIG. 13: The ecosystem surrounding a mobile inhaler with artificial intelligence capabilities with a wearable device and the associated dataflows, in accordance with some embodiments of the present disclosure;

FIG. 14 shows a perspective view of an illustrative inhaler with a removable front cover, in accordance with some embodiments of the present disclosure;

FIG. 15 shows a perspective view of the inside structure of the illustrative inhaler of FIG. 14, in accordance with some embodiments of the present disclosure;

FIG. 16 shows a perspective view of the inside structure of the illustrative inhaler of FIG. 14 without the electric motor to reveal the structural elements behind the electric motor, with a rack and pinion transmission mechanism, in accordance with some embodiments of the present disclosure;

FIG. 17 shows a side view of the inside structure of the illustrative inhaler of FIG. 14 with a rack and pinion transmission mechanism, without the electric motor to reveal the structural elements behind the electric motor, in accordance with some embodiments of the present disclosure;

FIG. 18 shows a cross-sectional view of an illustrative capsule, in accordance with some embodiments of the present disclosure;

FIG. 19 shows a perspective view of the illustrative capsule of FIG. 18, in accordance with some embodiments of the present disclosure;

FIG. 20 shows a side view of the illustrative capsule of FIG. 18 fitted into a membrane holder, in accordance with some embodiments of the present disclosure;

FIG. 21 shows a perspective view of the illustrative capsule of FIG. 18 fitted into a membrane holder, in accordance with some embodiments of the present disclosure;

FIG. 22 shows a cross-sectional view of the illustrative capsule of FIG. 18 fitted into a membrane holder, in accordance with some embodiments of the present disclosure;

FIG. 23 shows a cross-sectional view of the illustrative capsule of FIG. 18 coupled to a screw-type transmission mechanism, in accordance with some embodiments of the present disclosure; and

FIG. 24 is a flowchart of an illustrative process for controlling a mobile inhaler, in accordance with some embodiments of the present disclosure.

### DETAILED DESCRIPTION

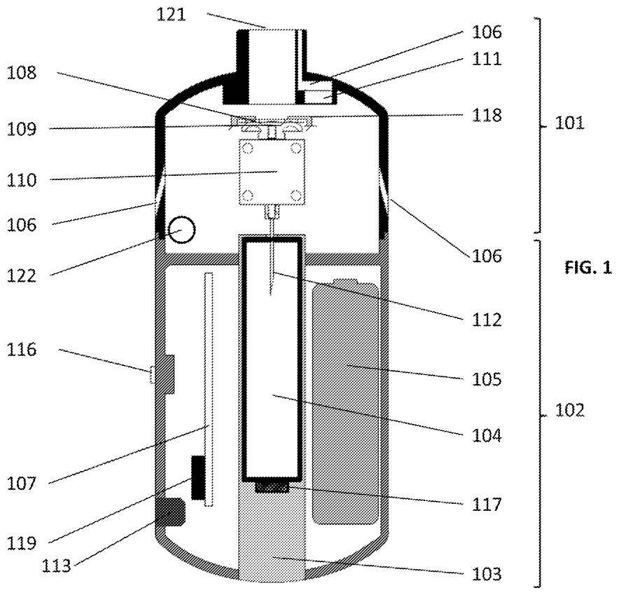

FIG. 1 shows a cross-section of an illustrative apparatus with a capsule inserted, in accordance with some embodiments of the present disclosure. In some embodiments, the apparatus includes:

Mouthpiece 101 having optional closable hermetic cover 121;

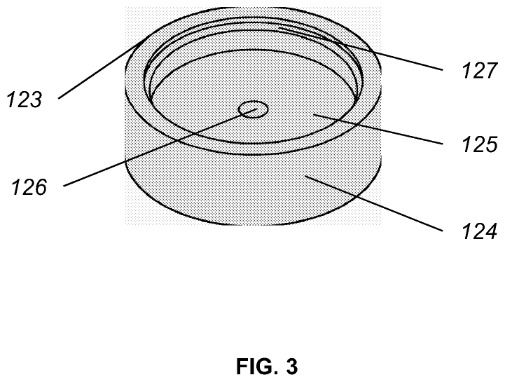

Body 102 with bottom aperture 103 for insertion of liquid container 104, battery 105 (e.g., or battery 301 of FIG. 3), or both. Battery 105 (e.g., or battery 301 of FIG. 3) can be either replaceable or rechargeable;

Air ducts 106 that pass-through mouthpiece 101, allowing air to be drawn from the outside (e.g., outside of mouthpiece 101 and body 102) and into mouthpiece 101, intermingling with the aerosol being generated from vibrating mesh membrane 108;

Electronic board or central processing unit (CPU) 107, including power converting unit 304 for membrane driving, the micro-pump 110 driver circuits, sensor drivers, controls, logic and communication interfaces;

Vibrating mesh membrane 108, which is actuated by a piezoelectric ring (e.g., the piezoelectric ring may be integrated as part of vibrating mesh membrane 108) located under membrane fixer block 118, and adjacent to liquid membrane chamber 109. In some embodiments of the apparatus, vibrating mesh membrane 108 can be activated by ultrasonic and/or electronic means (e.g., the piezoelectric ring, as controlled by control circuitry). In some embodiments of the apparatus, holes of vibrating mesh membrane 108 can have a uniform diameter while in some embodiments, the holes of vibrating mesh membrane 108 may have different diameters;

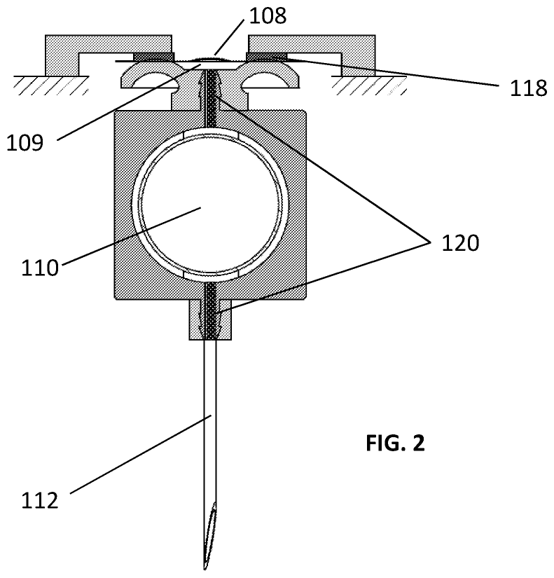

Micro-pump 110, with connecting pipes 120 (e.g., shown in FIG. 2), which couple needle 112 to micro-pump 110, and also couple micro-pump 110 to liquid membrane chamber 109;

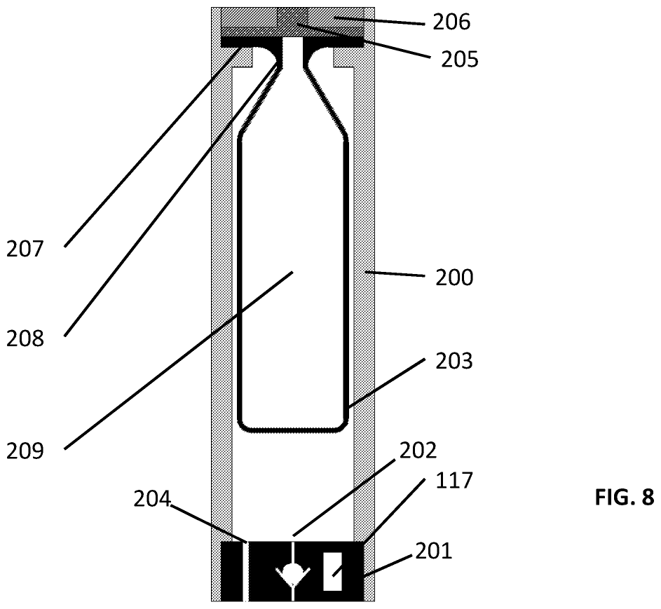

Air flow sensors 111 (e.g., similar to air flow sensor 303 of FIGS. 8), and 122 (e.g., similar to air flow sensor 312 of FIG. 3) for monitoring the air flow into the mouthpiece; and

Needle 112, which couples micro-pump 110 to liquid container 104 (e.g., which be or include a capsule), such that liquid from the liquid container 104 may reach micro-pump 110.

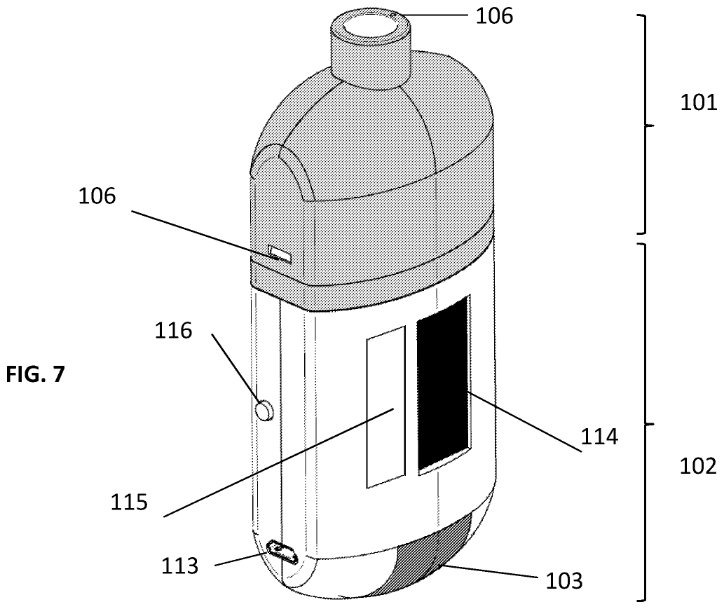

FIG. 7 is a 3-D drawing of the external image of the apparatus of FIG. 1. The following features are illustratively shown in FIG. 7:

Air ducts 106 for allowing external air to mix with generated aerosol within mouthpiece 101 (e.g., when mouthpiece 101 is under suction);

USB connector 113 (e.g., a mini-USB input, micro-USB input, or other USB input), as illustratively depicted in FIG. 7, for battery recharging and/or communication exchange between an external receiving device, and electronic board or CPU 107. In some embodiments, the external receiving device can be one of the following: a computer, iPhone, or personal digital assistant (PDA) type device.

User interface display 114 configured as a readout of data from electronic board or CPU 107 and data input by a user. The user interface display can include any suitable features, including for example, a touchscreen (e.g., configured to receive haptic input from a user);

Personal identification recognition means 115, which may include, for example, a fingerprint recognition sensor, biometric scanner, or both for user recognition, age determination, and safety;

Bottom aperture 103 configured for allowing exchange of the liquid container 104; and

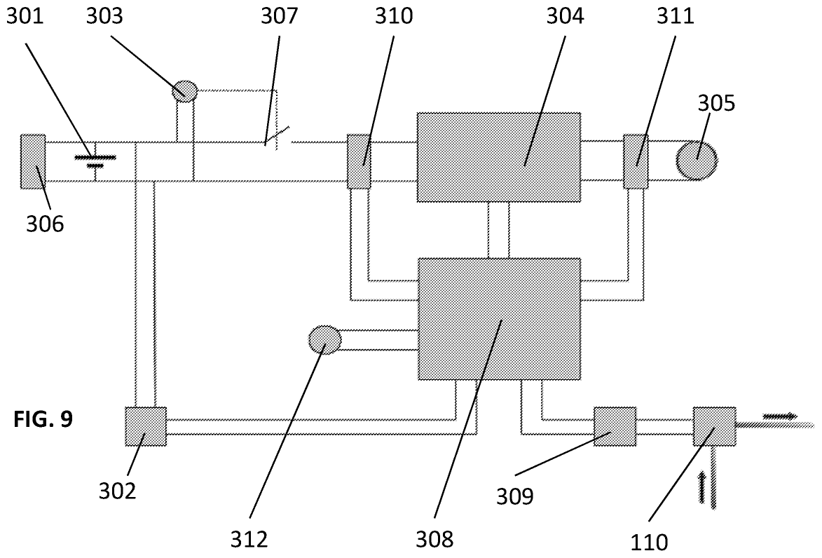

External on-and-off button 116 (e.g., or button 307 of FIG. 9). In some embodiments, further optional buttons could be included to, for example, control the generation and release of aerosol amounts (not shown).

FIG. 2 is an exploded view of micro-pump 110 and needle 112, membrane chamber 109, piezoelectric ring, membrane, and membrane fixer blocks in cross-section, in accordance with some embodiments of the present disclosure.

Micro-pump 110 draws liquid from liquid container 104 via needle 112 in a controlled and measurable manner and pushes it through connecting pipe 120 toward liquid membrane chamber 109. In some embodiments, micro-pump 110 allows the control and evaluation of the quantity of liquid to be nebulized, based on the known and controllable flow rate of micro-pump 110 itself (e.g., micro-pump 110 may be calibrated).

In some embodiments (not shown), micro-pump 110 is a piezoelectric-actuated micro-pump, composed of a micro-chamber, two non-return micro-valves for input and output flow of the liquid, and a piezoelectric component. The piezoelectric component moves forward and backward (e.g., vibrates) depending on the applied voltage waveform (e.g., having an amplitude and frequency spectrum), filling and emptying the micro-chamber through the micro-valves. In some such embodiments, the flow rate of micro-pump 110 is dependent on the frequency at which the pumping cycles are repeated, and/or on the voltage at which the piezoelectric component is actuated.

In some embodiments (not shown), micro-pump 110 can be replaced by a controlled micro-valve (e.g., a controllable flow restriction). In some such embodiments, the micro-valve is designed to release the liquid from the liquid container 104 in a controlled and measurable way. In this case, the liquid in the liquid container 104 must be stored under a sufficient pressure (e.g., since there is no micro-pump to boost the pressure), that allows total evacuation of the liquid from the liquid container 104 to the liquid membrane chamber 109.

In some embodiments (not shown), micro-pump 110 can be complemented by a micro-valve that helps to control the liquid flow from the liquid container 104 to the liquid membrane chamber 109. In some embodiments (not shown), a controllable micro-valve can be inserted also for better control of the back pressure from the liquid membrane chamber 109 toward the micro-pump 110.

In some embodiments, vibrating mesh membrane 108 is anchored to vibrating mesh membrane holder 123 in such a way, that the membrane can vibrate with minimum mechanical constraint. By anchoring the vibrating mesh membrane 108 in a manner that minimizes the mechanical constraint, it allows the vibrating mesh membrane 108 to operate at its maximum potential (e.g., with regard to frequency and amplitude).

Vibrating mesh membrane holder 123 is located below vibrating mesh membrane 108 and liquid membrane chamber 109 and serves to house vibrating mesh membrane 108 and the liquid membrane chamber 109. In addition to providing the anchoring system whereby allowing vibrating mesh membrane 108 to operate at its optimal potential (e.g., in terms of frequency and amplitude, such as a resonance), vibrating mesh membrane holder 123 also serves to prevent any leakage of the liquid being held in liquid membrane chamber 109. Further details of vibrating mesh membranes, piezoelectric ring actuators (e.g., ultrasonic vibrators) are described in U.S. patent application Ser. No. 15/770,735 filed on Apr. 24, 2018, titled “A Mobile Inhaler and a Container for Using Therewith,” which is hereby incorporated by reference herein in its entirety. For example, vibrating mesh membrane 108 may include a disc shape, with a central dome-shaped region having a plurality of holes arranged in the center of a ring-shaped piezoelectric vibration generator, which is electrically coupled to control circuitry.

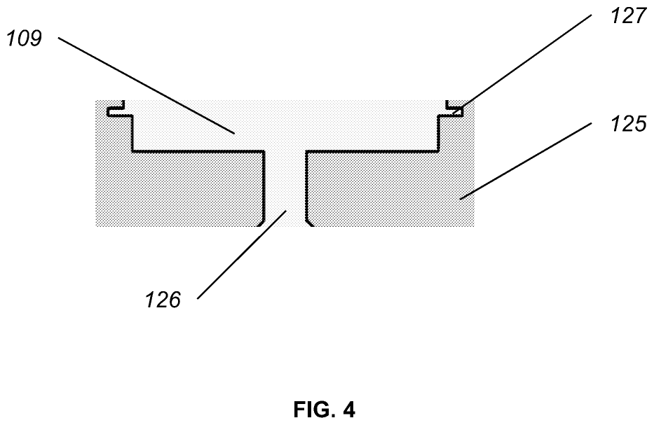

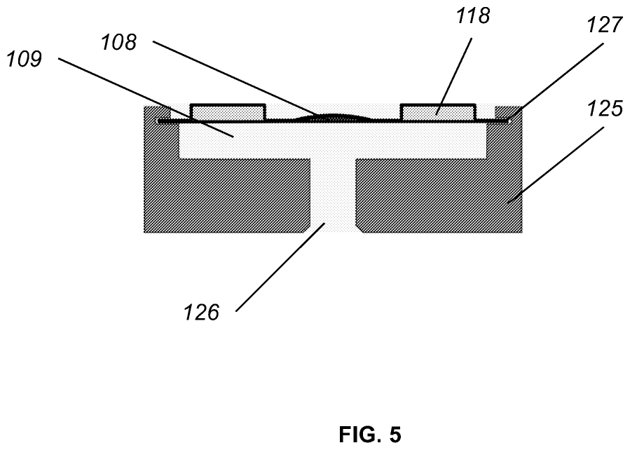

Vibrating mesh membrane holder 123 is illustratively shown in FIG. 3, and its cross-section is illustratively shown in FIGS. 4-5, in accordance with some embodiments of the present disclosure. Vibrating mesh membrane holder 123 includes an external chamber having vertical walls 124 and bottom floor 125 that includes hole 126. Hole 126 is formed in such a way that, for example, it can be directly inserted onto connecting pipe 120 that couples micro-pump 110 to liquid membrane chamber 109. This connection allows for the conveying of liquid from the capsule and at the same time acts as a gasket (e.g., a liquid-tight seal). In some embodiments, (not represented in the drawings), a second gasket can be added to hole 126, in order to assure the system is hermetic in all positions.

Vibrating mesh membrane holder 123 has an upper opening at the top where the membrane and piezoelectric ring are inserted and fixed into groove 127. FIG. 4 shows a cross-section of the vibrating mesh membrane holder 123 shows the vertical walls 125, which includes on the internal walls groove 127 running horizontally around the internal diameter of vibrating mesh membrane holder 123. The width of the groove 127 is approximately equal to the thickness of the outer edge of the vibrating mesh membrane 108 and piezoelectric ring, such this structure can be inserted into the groove. Groove 127 is designed to hold vibrating mesh membrane 108, ensuring hermeticity to the attachment.

In some embodiments, groove 127 can be complemented with, or in the alternative replaced by, a means for attaching the outer edge of vibrating mesh membrane 108 can be attached to the internal wall of vibrating mesh membrane holder 123 by any suitable “attaching means”, for example, a silicone paste, a glue, or any equivalent means.

Vibrating mesh membrane holder 123 is designed such that the coupling of vibrating mesh membrane 108, including the piezoelectric ring, and the electronic board or CPU 107 can be accommodated without modification to vibrating mesh membrane holder 123, thereby ensuring that vibrating mesh membrane holder 123 is leak proof.

The material with which the holder is fabricated must be such that the constraint (e.g., force or material stress) exerted on the outer edge of the membrane is minimized or otherwise does not damage the membrane. For example, other than adhesive forces, there may be little to no preload stress on vibrating mesh membrane 108.

In some embodiments, the material used to fabricate vibrating mesh membrane holder 123 is relatively soft, but still stiff enough to hold the vibrating mesh membrane 108 in a steady position, that ensures repeatable vibration of the vibrating mesh membrane holder 123. In some embodiments, the material used to fabricate vibrating mesh membrane holder 123 must also be hermetic and leak-proof with respect to the liquid contained in vibrating mesh membrane holder 123.

In some embodiments, vibrating mesh membrane holder 123 is fabricated using several different fabrication technologies including, for example, Fused Deposition Modelling (FDM), Fused Filament Fabrication (FFF), 3D printing technologies, any other suitable technologies, or any suitable combination thereof.

In some embodiments, vibrating mesh membrane holder 123 is fabricated using several different fabrication materials including, for example, Thermo Plastic Elastomer (TPE), Thermo Plastic Polyurethane (TPU), Acrylonitrile Butadiene Styrene (ABS), Poly Lactic Acid (PLA), any other suitable materials, or any suitable combination thereof.

In some embodiments, vibrating mesh membrane holder 123 can be fabricated by stereolithography, for example, using resins with suitable degrees of elasticity that achieve the described properties once the resin is polymerized. In some embodiments, molding and casting procedures are used to fabricate vibrating mesh membrane holder 123. In some embodiments, vibrating mesh membrane holder 123 are fabricated using several different fabrication technologies including, with the described properties. In some embodiments, the volume of vibrating mesh membrane holder 123 is selected in such a way that optimum nebulization of the liquid by the membrane is allowed.

In some embodiments, the pressure of the liquid in vibrating mesh membrane holder 123 must not exceed a threshold that reduces the mobility of the vibrating mesh membrane 108, reducing also the amplitude of the vibrations and therefore the effectiveness of the nebulization. For example, large pressure forces may impact performance of vibrating mesh membrane 108 by reducing the deformation amplitude.

In some embodiments, the quantity of liquid in the vibrating mesh membrane holder 123 cannot be reduced by the nebulization in such a way, that the level of the liquid is not sufficient to keep the vibrating mesh membrane 108 in contact with liquid. For example, liquid voids (e.g., pockets of gas without liquid phase material) near vibrating mesh membrane 108 may be undesired during operation.

In some embodiments, the optimum quantity and pressure of the liquid can be regulated by matching the liquid flow rate of micro-pump 110 into vibrating mesh membrane holder 123 with the nebulization rate of the vibrating mesh membrane 108. In some embodiments, the liquid pressure inside vibrating mesh membrane holder 123 can be evaluated and monitored by means of a pressure sensor or an equivalent device, which is coupled to the holder. In some embodiments, the liquid pressure value is fed seamlessly to the microcontroller which in turn instantly adjusts the flow rate of micro-pump 110, the nebulization rate of vibrating mesh membrane 108, or both. This is controlled, monitored and adjusted by means of the electronics.

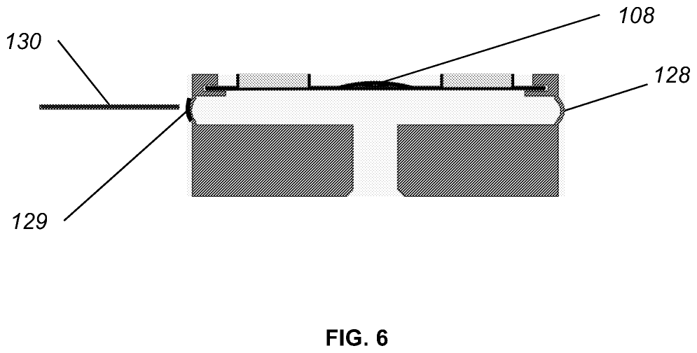

In some embodiments (e.g., as shown in FIG. 6), a portion of vertical walls 124 of the vibrating mesh membrane holder 123 can be made to include a section of flexible or soft wall 128. The inclusion of a section of flexible or soft wall 128 allows for the volume of vibrating mesh membrane holder 123 to increase or decrease as the value of the pressure of the liquid in the vibrating mesh membrane holder 123 falls outside of a predetermined pression range, due for example to a mismatch between the flow rate of the micro-pump and nebulization rate of the vibrating mesh membrane 108. For example, if micro-pump 110 supplies more liquid to vibrating mesh membrane holder 123 than is nebulized by vibrating mesh membrane 108 during a suitable time interval (e.g., depending upon the accumulation volume and flow rates), the volume contained within flexible or soft wall 128 may increase accordingly due to deformation caused by pressure forces.

The expansion or reduction of the volume of liquid within vibrating mesh membrane holder 123 helps reduce the variation of the pressure exercised by the liquid on the walls, helping to maintain the pressure of the liquid itself within a set range, and consequently allowing for the effective nebulization by vibrating mesh membrane 108. For example, flexible or soft walls 128 may allow for relatively increased operating ranges (e.g., in terms of flow rate, fluid properties, or transient behavior) of micro-pump 110, vibrating mesh membrane 108 or both.

In some embodiments, (e.g., as shown in FIG. 6) the addition of a pressure sensor 129 to the section of the flexible or soft wall 128 of the vibrating mesh membrane holder 123 can be implemented in such a way, that an electrical contact is made when the volume of the vibrating mesh membrane holder 123 exceeds a predetermined volume. In some embodiments, the pressure sensor 129 is a metallic wire or strip. In an illustrative example, pressure sensor 129 may function as a pressure switch (e.g., as a two-state device).

In some embodiments, probe 130 is arranged at a given distance to pressure sensor 129 in such a way that an electrical contact is made between sensor 129 and probe 130 when flexible or soft walls 128 of vibrating mesh membrane holder 123 are stretched beyond a predetermined deformation. In some embodiments, probe 130 is a metallic wire or strip (e.g., or any other suitable electrically conductive element).

When the probe 130 and pressure sensor 129 are in contact with each other, an electrical contact is detected by the microcontroller, that reacts by adjusting the flow rate of the micro-pump and nebulization rate of vibrating mesh membrane 108 in such a way that the quantity of liquid in the vibrating mesh membrane holder 123 is reduced, and the volume of the vibrating mesh membrane holder 123 subsequently decreases, which in turn opens the contact between the connectors. In some embodiments, the electrical contact between probe 130 and pressure sensor 129 may be included in a circuit that directly controls micro-pump 110, vibrating mesh membrane 108, or both (e.g., by controlling a relay). In some embodiments, the electrical contact between probe 130 and pressure sensor 129 may be monitored by the microcontroller, which may, optionally also based on other criteria, control micro-pump 110, vibrating mesh membrane 108, or both.

In some embodiments, as illustrated, hermetic cover 121 is optionally included over the mouthpiece. Hermetic cover 121 may be opened and closed by a button by the user, or manually opened and automatically closed by the electronic board or CPU 107 by means of an actuator, after a given time interval after the last operation of the vibrating mesh membrane 108. Hermetic cover 121 prevents prolonged contact between the liquid and the external air through the holes in the vibrating mesh membrane 108 (e.g., when the nebulizer is not in use). Hermetic cover 121 provides a hygienic and safe way of transporting the apparatus (e.g., preventing external air or gas from contacting the internal portion of the apparatus).

Liquid container 104 can be inserted into body 102 from bottom aperture 103. In some embodiments, the liquid container 104 is a disposable item to be replaced at the occurrence of an event (e.g., a time limit, when the liquid container 104 is empty, the liquid container 104 is to be swapped to change liquid) and contains the liquid solution to be consumed by the user.

Liquid container 104 must be connected to the micro-pump 110 to deliver the liquid solution from one of its ends, referred to herein as a “front” end (e.g., near the micro-pump); the other end is referred to herein as “rear” end (e.g., away from the micro-pump).

In accordance with the present disclosure, the liquid container 104 is configured as leak proof, during storage, and during and after its use within the apparatus. In some embodiments, the liquid container 104 includes (as shown in FIG. 8) capsule body 200, rear plug 201, deformable balloon 203, one-way valve 202, pressurizing air duct 204, front seal 205, and front plug 206.

Capsule body 200 serves to encase the other components of the liquid container 104. Liquid container 104 contains all the parts and, moreover, it supports and isolates, based on mechanical supports. For example, rear plug 201, front seal 205 and front plug 206, deformable balloon 203 (e.g., a bladder). The volume between capsule body 200 and deformable balloon 203, hereinafter called “inner volume,” can be pressurized or not. Capsule body 200 can be made of any suitable type of material. In some embodiments, the liquid container 104 is made of a light-weight plastic. In some such embodiments, the light-weight plastic is biodegradable.

Rear plug 201 may be assembled to the rear end of capsule body 200, or, in some embodiments, it can be realized in one piece with capsule body 200 (e.g., capsule body 200 includes rear plug 201). In some embodiments (e.g., if the inner volume is pressurized), rear plug 201 can be equipped with one-way valve 202. Otherwise, rear plug 201 may include very small hole(s), or be completely open, to maintain the same external pressure (e.g., outside of capsule body 200) in the inner volume.

Deformable balloon 203 may be subdivided into three parts: balloon rim 207, balloon reservoir 209, and optional balloon neck 208. The internal volume of deformable balloon 203 is hereinafter referred to as balloon reservoir 209. Balloon reservoir 209 is filled with the liquid solution and avoids leaks towards the inner volume (e.g., through deformable balloon 203). Balloon rim 207 allows deformable balloon 203 to be fixed to capsule body 200. For example, the face of balloon rim 207, directed toward balloon reservoir 209, is called the lower face. The opposite face (e.g., facing up in FIG. 8) is called upper face.

One-way valve 202 is configured to allow pressurization of balloon reservoir 209 during the assembling process, and to maintain an overpressure (e.g., a pressure in the inner volume that is greater than a pressure at the outlet of liquid container 104) during the whole life cycle of liquid container 104.

Pressurizing air duct 204 is configured to allow pressurized gas to apply pressure on the exterior of deformable balloon 203 to cause dispensing of liquid.

Front seal 205 is configured to close deformable balloon 203 off from the exterior environment of capsule body 200. Front seal 205 is in contact with the upper face of balloon rim 207 and is directly accessible from outside of liquid container 104. Front seal 205 is configured to allow the passage of needle 112, to deliver the liquid solution from deformable balloon 203 to outside of liquid container 104. In some embodiments (not shown), needle 112 and front plug 206 can be a single piece. In some embodiments, front plug 206 is an independent component from needle 112.

Front plug 206 is assembled to the front end of the capsule body 200. Front plug 206 together with front seal 205 ensures the sealing of liquid container 104.

More specifically, in some embodiments, deformable balloon 203 and front seal 205 enclose the volume to be filled with the liquid solution. Capsule body 200, rear plug 201, and front plug 206 form the complete external case, and can be arranged by different assembly modes according to the technological and assembling process needs.

To deliver the liquid solution from liquid container 104, needle 112 must pass through the front end of liquid container 104. Front seal 205 must prevent leakage of the liquid solution outside of balloon reservoir 209 and prevent air from entering inside balloon reservoir 209 before, during and after the insertion or removal of the liquid container 104 into the mouthpiece 101, body 102, or both, of the apparatus.

Needle 112 is configured to enter balloon reservoir 209 of deformable balloon 203, which is filled with the liquid solution. A difference of pressure between the ends of needle 112 is used to draw the liquid from liquid container 104 into liquid membrane chamber 109. Deformable balloon 203 must change its volume according to the quantity of the remaining liquid solution, until the emptying of the liquid container 104. For example, as liquid solution is consumed as aerosol by a user, balloon reservoir 209 is depleted of liquid and accordingly reduces in volume.

The liquid contained in the liquid container 104 may include compounds that are suitable for, for example, nicotine replacement therapy, or alternatively where nicotine is partly or fully replaced by other chemical compounds, for example any miscible pharmaceutical drug or nutrient. There are several benefits of using the vibrating mesh technology as described by the present disclosure for delivering liquid nicotine, for example:
Nicotine salt is more effective than nicotine freebase in terms of uptake in the bloodstream at low temperatures,
Nicotine salt (protonated) is less addictive than nicotine freebase (ion),
No transformation of liquid composition with the claimed vibrating mesh technology in the vaporization process, including no heat-induced chemical changes (e.g., pyrolysis or other chemical changes),
Optimal targeted delivery of nicotine salts and freebase nicotine to the lung, by
droplet size distribution and control of droplet size and quality.
Combination of nicotine salts (less addictive) with freebase nicotine (more addictive) represent considerable advantage for de-addiction and nicotine replacement therapies through vibrating mesh technology metered-dosage capabilities.

In some embodiments, a liquid composition which includes nicotine, could be characterized as follows:
Nicotine Volume: 0.5% of total volume (100 mg/ml) for a 0.5 mg/ml Nicotine concentration to 20% of total volume (100 mg/ml) for a 20 mg/ml Nicotine concentration. 0% of total volume for a 0 mg/ml Nicotine concentration.
Nicotine Types & Splits: Freebase Nicotine from 0% to 100% (99.9 mg/ml at a pH of 8.5 to 9.5) of the Nicotine Volume combined with Nicotine salts from 0% to 100% (100 mg/ml at a pH of 4 to 5) of the Nicotine Volume.
Aroma Volume: 0.5% to 25% of total volume (Aromatic compounds dissolved in propylene glycol, propylene glycol is a solvent and a stabiliser of aroma which allow miscibility with water).
Distilled Water Volume: 20% to 90% of total volume.
Vanillin Volume: 2% to 10% of total volume. Vanillin acts as an agent that smoothens the nicotine itch or harshness in the throat, vanillin is dissolved in propylene glycol.

Some embodiments of the present disclosure increase the efficacy and ease of delivering medicinal compounds or drugs by a novel delivery system that delivers compounds to the deep lung by metered-dosaging regulation.

The use of vibrating mesh technology allows deep-lung penetration to be achieved for several classes of drugs. The following list of illustrative drug classes may be suitable for use in liquid compounds to be used in accordance with the present disclosure (e.g., to obviate deficiencies seen with existing drug delivery routes and administration techniques):
Drugs of the autonomic nervous system such as, for example, cholinergics and adrenergics;
Drugs of the central nervous system (CNS) such as, for example, CNS stimulants, neuroleptics, anxiolytics, hypnotics, sedatives, opioids analgesics and antagonists, anesthetics, drugs to treat Parkinson disease, epilepsy, depression and anxiety disorder, psychosis and mania;
Drugs of the cardiovascular system such as, for example, anti-anginals, congestive heart failures, anti-arrhythmics, anti-hypertensives, anti-hyper lipidemics;
Drugs acting on blood;
Drugs of the gastro intestinal tract such as, for example: drugs addressing peptic ulcers, anti-diarrheals, anti-motility, anti-constipates, drugs addressing inflammatory bowel diseases, anti-emetics;
Drugs of the respiratory system such as, for example, drugs addressing asthma and other respiratory diseases;
Drugs addressing the renal function such as, for example diuretics;
Chemotherapeutic drugs such as, for example, anti-bacterials, anti-amoebics, anti-fungals, anti-malarials, anti-virals, anti-cancers;
Drugs of inflammation and pyrexia such as, for example, anti-inflammatory, analgesics and anti-pyratics, autacoids and autacoid antagonists;
Drugs of immunomodulation such as, for example immunosuppressants;
Drugs of hormonal disorders such as, for example, anti-diabetic drugs, steroid hormones;
Drugs directed to Dermatology; and
Drugs directed to other diseases and systems to treat obesity, erectile disfunction, or HIV-AIDS, for example.

Some embodiments of the present disclosure increase the efficacy and ease of delivering medicinal compounds or drugs to the lung. Lungs are an attractive target for the pulmonary administration of active pharmaceutical ingredients or APIs in the form of various drug delivery systems. Additionally, this route offers many advantages over conventional oral administration, such as a high surface area with rapid absorption due to high vascularization and circumvention of the first pass effect. Pulmonary drug delivery offers several advantages compared with intravenous, oral, buccal, transdermal, vaginal, anal, nasal or ocular administration:
A noninvasive ‘hypodermic needle-free’ delivery system;
Wide range of substances from small molecules to very large proteins;
Enormous absorptive surface area (100 m2) and a highly permeable membrane (0.2-0.7 mm thickness) in the alveolar region;
Large molecules with very low absorption rates can be absorbed in significant quantities; the slow muco-ciliary clearance in the lung periphery results in prolonged residency in the lung;
A less harsh, low enzymatic environment;
The bypass of hepatic first-pass metabolism;
Reproducible absorption kinetics; and
Independent of dietary complications, extracellular enzymes and interpatient metabolic differences that affect gastrointestinal absorption.

This selectivity allows targeted drug delivery and, hence, reduces the side effects.

Liquid container 104 can optionally be equipped with identification means 117 (e.g., see FIG. 8). Identification means 117 can include, for example, a RFID or Bluetooth low energy tag, a bar code, any other suitable equivalent identification, or any combination thereof. Identification means 117 is recognized by corresponding identification means sensor 119, which is connected to electronic board or CPU 107. In some embodiments, information included (e.g., stored in memory or as an identifiable tag) in identification means 117 can include information about the contents of liquid container 104. For example, the indication of the type of liquid, a detailed or partial composition profile of the liquid (e.g., the liquid's nicotine content), the total volume of the liquid, the allowed type of usage for a specific liquid container 104, and/or any other suitable information pertaining to the liquid and/or the liquid container 104 itself In another embodiment, identification means 117 can optionally be used to detect the insertion and/or removal of liquid container 104 into or from body 102 of the apparatus.

An illustrative electronics system or “processing equipment” of the apparatus is shown in FIG. 9. Battery 301 (e.g., which may be similar to battery 105 of FIG. 1) supplies DC voltage to voltage regulator 302, on-off air flow sensor 303 (e.g., which may be similar to on-off air flow sensor 111 of FIG. 1), and power converting unit 304. Power converting unit 304 transfers electric power to vibrating mesh membrane 305 (e.g., vibrating mesh 108 of FIG. 1). Battery 301 (e.g., which may be similar to battery 105 of FIG. 1) is recharged through the on-off air flow sensor 303 (e.g., which may be similar to on-off air flow sensor 111 of FIG. 1) by USB connector 306 (e.g., which may be similar to USB connector 113 of FIG. 1), that can be used also as a communication port for data transfer to and from an external logic unit. On-off air flow sensor 303 also controls the connection between the battery 301 or 105 and the power converting unit 304 through an on-and-off switch 307. For example, as on-off air flow sensor 303 senses airflow, and when the air flow is above a threshold (e.g., a voltage or current from the sensing element exceeds a threshold), switch 307 may be closed. To illustrate, referencing FIG. 1, on-off air flow sensor 111 may sense airflow through mouthpiece 101 from user inhalation. Further, because the airflow is present, and switch 307 is closed, aerosol generated at vibrating mesh membrane 305 (e.g., which now may be powered) may be entrained by the air (e.g., to aid in transport of the aerosol droplets for inhalation). In some embodiments, on-off air flow sensor 303 and switch 307 are integrated as a single component and may function as a flow switch.

In some embodiments, switch 307 includes a power metal-oxide-semiconductor field-effect transistor (MOSFET) device or equivalent (e.g., any suitable mechanical switch, relay, solid state component, or combination thereof).

The electrical connection (e.g., from battery 301 to input detector 310 and other suitable components of the electrical system) is activated by switch 307 only when air is drawn through the mouthpiece (e.g., mouthpiece 101 of FIG. 1) by the user, as detected by the on-off air flow sensor 303 and 111. Microcontroller unit 308 and 402 is always on (e.g., when power is available), for fast reaction to the on-off sensor 303 (e.g., to minimize boot-up time). Once the on-off air flow sensor 303 and 111 is activated (e.g., and switch 307 is closed), the microcontroller unit 308 and 402 starts the power conversion (i.e., by power converting unit 304) from the battery 301 and 105 to the vibrating mesh membrane 305 and 108 and to the micro-pump driving unit 309. In both cases, power converting unit 304 converts the input DC power into an alternating voltage suitable for vibrating mesh membrane 305, with controlled amplitude, waveform shape and frequency. The waveform can be a sinusoid, a square wave, a staircase wave, any other similar waveform, or any suitable combination thereof. For example, a staircase waveform is usually alternating between a positive level, a zero level and a negative level. The fraction of time when the waveform is non-zero (e.g., the duty cycle) can be varied from almost zero to almost one. All these waveforms can be continuous in time or can be generated in bursts.

Input detector 310 and/or output detector 311 are optionally present and serve to sample the current (e.g., in the DC or AC leads, respectively), the voltage (e.g., in the DC or AC leads, respectively), or both. If one or both of detectors 310 and 311 are present, the detected quantities are read by microcontroller unit 308. Microcontroller unit 308 sets the frequency of the waveform in such a way that the output voltage is maximized, the input DC current or the output alternating current amplitude is minimized, or in such a way that one or both are optimized after a prescribed algorithm. In this way vibrating mesh membrane 305 or 108 is operated at, or near, its resonant frequency, and the energy is most efficiently used for activating one or more vibration modes of vibrating mesh membrane 305.

In some embodiments, if vibrating mesh membrane 305 or 108 is operated at a frequency different from the resonant frequency, for example, the generated vibrations are reduced with respect to the same applied voltage waveform at the resonant frequency. The control of the frequency can be used for transferring the energy from battery 301 or 105 to vibrating mesh membrane 305 or 108 most efficiently or can be used to vary the energy transferred to vibrating mesh membrane 305 or 108.

Intensity flow sensor 312 and 122 can be optionally present and connected to one or more of air ducts 106 on the side of the mouthpiece. Preferably, intensity flow sensor 312 and 122 is located adjacent to one of air ducts 106 (e.g., where flow velocities may be relatively higher) on the side of the mouthpiece.

Intensity flow sensor 312 and 122 evaluates the intensity of the air flow drawn by the user from the mouthpiece. Examples of practical implementations of the intensity flow sensor 312 and 122 can include: a microphone, a piezoelectric pressure sensor or a temperature sensor. In some embodiments using the temperature sensor, an optional second sensor is placed in body 102 of the apparatus and acts as a reference temperature sensor.

In some embodiments, optional wireless communication circuitry can be included in the apparatus, connected to microcontroller unit 308. For example, wireless communications circuitry may include a Bluetooth chip, WIFI circuitry for internet protocol-based communication, RFID circuitry, or similar communication circuitry and protocols, or any suitable combination thereof.

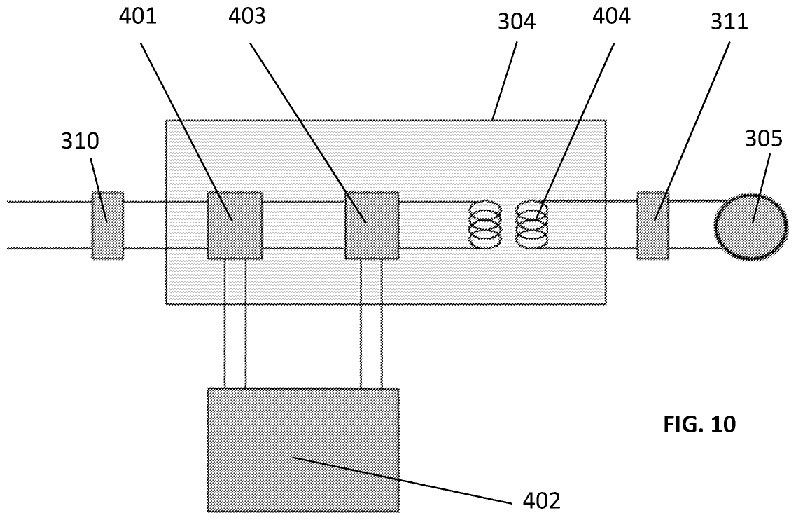

Power converting unit 304, shown in more detail in FIG. 10, can optionally include DC/DC converter 401, that increases or decreases the value of the DC voltage output to DC-AC converter 403, controlled by the microcontroller unit 402 and 308. DC/AC converter 403 generates an alternating voltage waveform as described hereinabove (e.g., suitable for generating ultrasonic waves in vibrating mesh membrane 108). A transformer 404 is optionally present, for increasing the amplitude of the alternating voltage waveform at the output. Transformer 404 may, in some embodiments, also provide isolation of the electrical input and output of power converting unit 304.

The value of the DC voltage (e.g., output from DC/DC converter 401) determines the amplitude of the generated alternating voltage waveform. The larger the amplitude, the higher the power transferred to the vibrating mesh membrane 108. If the alternating voltage waveform is generated in bursts, for example, the longer the burst with respect to the time when no burst is present, the higher the power transferred to vibrating mesh membrane 305. If the alternating voltage waveform is a staircase waveform, the higher the duty cycle (as described hereinabove), the higher the power transferred to the vibrating mesh membrane 305. Similarly, equivalent manipulations of the waveform can be applied, to increase or reduce the power transferred to the vibrating mesh membrane 305.

Any or all of the described means for the control of the power transferred to the vibrating mesh membrane 305 can be used to control the amount of aerosol (e.g., related to the liquid consumption rate) generated by the vibrating mesh membrane 305.

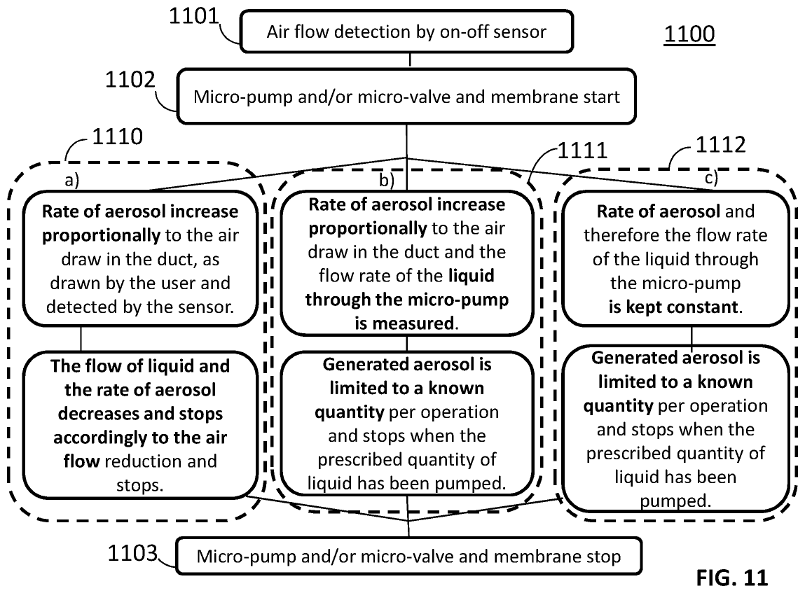

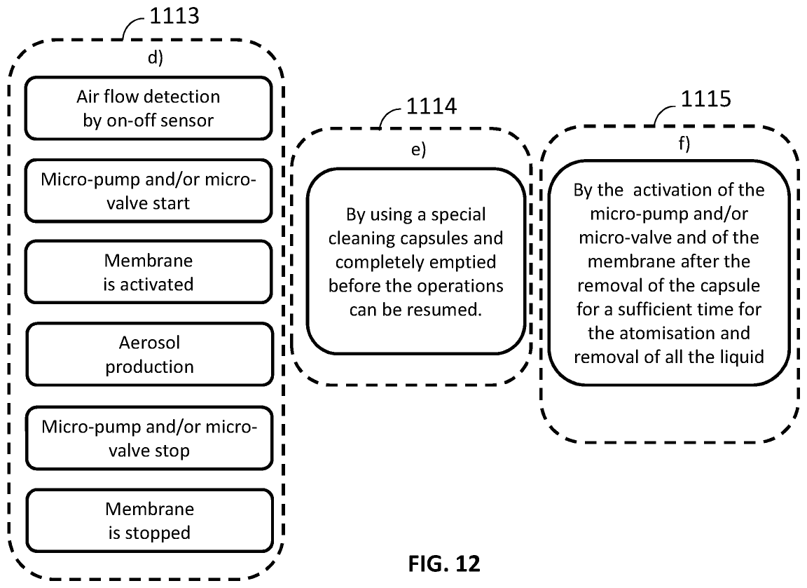

FIG. 11 shows illustrative process 1100 including operation modes of the apparatus, in accordance with some embodiments of the present disclosure. Operation modes 1110, 1111, and 1112 include illustrative examples of operation of the apparatus, which may be used independently or in combination. FIG. 12 shows additional operating modes 1113, 1114, and 1115, in accordance with some embodiments of the present disclosure. Any of operation modes 1110-1115 (e.g., also labeled as a-f), alone or in combination, may be used in accordance with the present disclosure. For example, an illustrative process for operating an apparatus may include step 1101, step 1102, any or all of operation modes 1110-1115, and step 1103.

Step 1101 includes detecting air flow using an on-off sensor (e.g., on-off air flow sensor 303). Step 1102 includes starting a micro-pump, micro-valve and membrane, or both. Step 1103 includes Step 1102 includes stopping a micro-pump, micro-valve and membrane, or both.

Operation mode 1110 of FIG. 11 includes generating aerosol in proportion to an amount of airflow (e.g., to keep the aerosol density per air volume near constant). For example, in some circumstances, during one inhalation, air flow may increase from zero to a maximum value, and then decrease back to zero. Operation mode 1110 includes micro-pump 110 and vibrating mesh membrane 108 starting when on-off air flow sensor 111 and 303 detects an air flow (e.g., above a threshold, or any nonzero value) through air duct 106 at the top of the mouthpiece, as drawn by the user via inhalation. In some embodiments, on-off air flow sensor 303 may be configured to differentiate between inhalation and exhalation of a user. The flow of liquid from liquid container 104 to liquid membrane chamber 109, and the rate of aerosol generation by the vibrating mesh membrane 108, is regulated in such a way that it increases from a minimum quantity at the start of the operation, in response and proportionally to the air draw in air ducts 106 at the side of mouthpiece as drawn by the user, and as detected by the intensity of air flow sensor 122 or 312 in the vicinity of one of the two air ducts of the mouthpiece. The flow of liquid and the rate of aerosol generation then decreases and stops completely when the air flow through air ducts 106 at the top of the mouthpiece decreases and stops. The amount of liquid transferred from liquid container 104 to liquid membrane chamber 109 and the amount of aerosol generated by vibrating mesh membrane 108 are determined by the electronic board or CPU 107 in the manner described above through the control of the flow through micro-pump 110 and of the energy transferred to vibrating mesh membrane 108.

Illustrative operation mode 1111 of FIG. 11 includes, for example, a known amount of liquid being atomized. For example, operation mode 1111 may be used to deliver a dose or other discrete amount of liquid in total to a user. Operation mode 1111 includes limiting the amount of liquid supplied to vibrating mesh membrane 108, and therefore the amount of generated aerosol, to a known quantity per operation, by measuring the flow rate of the liquid through micro-pump 110 and stopping the flow when the prescribed quantity has been pumped. In an illustrative example, operation modes 1110 and 1111 may be combined to deliver a predetermined total amount liquid at a controlled aerosol density (e.g., number of droplets per volume) in the air. In a further example, the processing equipment may integrate or otherwise perform numerical quadrature of a measured or estimated atomization rate or flow rate to determine the known amount.

Illustrative operation mode 1112 of FIG. 11 includes atomizing a known amount of liquid, with the atomization rate held constant. For example, operation mode 1112 may be used to deliver a dose or other discrete amount of liquid in total to a user, at a fixed liquid atomization rate (e.g., although delivery of liquid aerosol to the user's lungs may depend on instant air flow which may change). Operation mode 1112 may include maintaining a constant rate of aerosol generation by vibrating mesh membrane 108. Accordingly, the flow rate of the liquid through micro-pump 110 is maintained constant during the operation, until a predetermined quantity of liquid has been atomized, vaporized, or both.

Illustrative operation mode 1113 of FIG. 12 includes filling liquid membrane chamber 109 with liquid, and then sequentially atomizing the accumulated liquid until liquid membrane chamber 109 is empty. Operation mode 1113 includes actuating micro-pump 110 during a short-time interval before actuation of vibrating mesh membrane 108, in such a way to fill liquid membrane chamber 109 before the beginning of the nebulization. Then, vibrating mesh membrane 108 actuation is stopped during a second short-time interval after the end of the actuation of the micro-pump 110, in such a way that vibrating mesh membrane 108 empties liquid membrane chamber 109 before stopping. In this way, the liquid is completely removed from liquid membrane chamber 109, and therefore is absent in the next nebulization, not contaminating it with a foreign and unwanted substance (e.g., if a liquid container 104 is replaced and a different liquid is used). In some circumstances, accumulated liquid in the apparatus, outside of a liquid container 104 is minimized to prevent or reduce mixing of liquids of different capsules. In some embodiments, any of operation modes 1110, 1111, and 1112, or a combination thereof, are combined with operation mode 1113. For example, operation mode 1112 may be implemented, and vibrating mesh membrane 108 may be actuated for a short-time interval after micro-pump 110 is de-actuated to clear liquid membrane chamber 109 of liquid.

Illustrative operation mode 1114 FIG. 12 includes using a cleaning capsule (e.g., liquid container 104 which contains a cleaning liquid) to clean or otherwise purge liquid from portions of the apparatus outside of liquid container 104. For example, in some embodiments, for the apparatus to be operated after a given and prescribed number of uses (e.g., as described above in the context of operation modes 1110-1113), a self-cleaning protocol must be exercised. Operation mode 1114 includes inserting one or more special cleaning capsules (e.g., dedicated to cleaning rather than providing medication) to be inserted in the apparatus and emptied. In some embodiments, more than one cleaning capsule may be inserted (e.g., in a given and prescribed sequence), and completely emptied one after another before the operations as above (e.g., and of operation modes 1110-1113) can be resumed. In an illustrative example, a sequence of cleaning capsules, each containing similar or different liquid from the other of clean capsules, may be inserted and depleted after each nebulizing use of the apparatus by the user. Operation mode 1114 may be implemented to ensure a periodic cleaning of the apparatus, to keep it clean and efficient.

Illustrative operation mode 1115 of FIG. 12 includes purging liquid from the apparatus, after a capsule is removed. Operation mode 1115 requires activation of micro-pump 110 and of vibrating mesh membrane 108 after the removal of liquid container 104 from the body of the apparatus. The activation is maintained for a sufficient time for the atomization and removal of all the liquid contained in liquid container 104 and in connecting pipes 120 that connect liquid container 104 to micro-pump 110 and to liquid membrane chamber 109. Operation mode 1115 can be used in conjunction with all the previously described operation modes (e.g., any or all of operation modes 1110-1114 of FIGS. 11-12).

In an illustrative operation mode (not illustrated), personal identification recognition means 115 is configured to recognize the user via fingerprint recognition or biometric sensor, or via a required password or code, to be communicated through an existing communication interface such as, for example, a Bluetooth chip, WIFI circuitry for internet protocol-based communication, RFID circuitry, or similar communication circuitry and protocols. Additionally, identification means 117 can be used to set the quantities of liquid to be atomized, also in conjunction with the identification of the type of liquid in liquid container 104, and to control the timing and operation mode of the atomization. For example, identification means 117 can optionally be used for allowing the operation of vibrating mesh membrane 108 only when an authorized user, as defined in a preliminary procedure controlled by the logic in the electronic board or CPU 107. In a further example, this technique can be used in conjunction with all the previously described operation modes (e.g., operation modes 1110-1115 of FIGS. 11-12).

In some embodiments, the illustrative examples of operation modes diagramed in FIG. 11 and FIG. 12 do not require modifications of the circuitry or mechanics of the apparatus, but only a selection of the suitable succession of specified types of operations by the microcontroller unit. For example, in some embodiments, any of operation modes 1110-1115 of FIGS. 11-12 may be performed without changing hardware or circuitry of the apparatus (e.g., other than perhaps a capsule). This selection of operation mode or other operating characteristics (e.g., such as time intervals, AC frequency and amplitude, or other characteristics) can be fixed or can be changed by the user via the communication interfaces, or via external manual selectors, or via any other selection means.

In addition to providing a safer and more acceptable delivery mechanism for nicotine, for example, the present apparatus is well-suited to address the problem of low solubility and permeability that is achieved with existing routes of drug delivery (e.g., non-pulmonary routes). Ultimately, the selection of a solubility improving method depends on each specific drug's chemical properties, site of absorption, and required dosage form characteristics. For example, operation mode and operating characteristics may be selected based on the contents of a capsule 104 to achieve a desired delivery. To illustrate, some liquids may need to be delivered more slowly, or in lower doses, to prevent irritation to a user's respiratory tract. Various techniques may be used to enhance the solubility of poorly soluble drugs. These techniques include, for example, physical and chemical modifications of the drug, particle size reduction, crystal engineering, salt formation, solid dispersion, use of surfactant, complexation, etc. In some circumstances, however, these efforts to improve solubility have been modestly successful.

In some embodiments, the mobile inhaler can include artificial intelligence (AI) in order to provide additional medical benefits and treatments. AI is the ability of a computer or other machine to perform actions thought to require intelligence. More specifically, it involves the ability of a computer to perform operations analogous to learning and decision making in humans, as by an expert system, a program for CAD or CAM, or a program for the perception and recognition of shapes in computer vision systems. Among these actions are logical deduction and inference, creativity, the ability to make decisions based on past experience, insufficient information, conflicting information, or a combination thereof.

In the context of medical benefits and treatments, AI uses computer techniques to perform clinical diagnoses and suggest patient treatments. AI has the capability of detecting meaningful relationships in a dataset and has been widely used in many clinical situations to diagnose, treat, and predict medical outcomes and results.

AI is changing healthcare, and may be applied in the context of a mobile inhaler, in any suitable way such as, for example, managing data, performing repetitive tasks, treatment design, medication management, and health monitoring.

Managing Data: Compiling and analyzing information, for example, like medical records and other past histories, data management is the most widely used application of artificial intelligence and digital automation. Furthermore computing machines collect, store, re-format, and trace data to provide faster, more consistent access. For example, a computer or other suitable machine may be configured to manage medicals records, user information, or other data in connection with a mobile inhaler. In an illustrative example, a computing machine may store data from a plurality of medical records, and format the data for faster access.

Performing Repetitive Tasks: Analyzing data entries, and other mundane tasks can all be done faster and more accurately by devices that use AI. For example, a computer or other suitable machine may be configured to analyze, record, and manage user input in connection with a mobile inhaler.

Treatment Design: Artificial intelligence systems have been created to analyze data using—notes and reports from a patient's medical files, external research, and clinical expertise—to create customized treatment plans for individual patients. For example, a computer or other suitable machine may be configured to generate a customized treatment plan for a user with regard to a mobile inhaler. In some embodiments, a computer or other machine may determine a treatment plan, alter a treatment plan, or determine to end a treatment plan based on gathered information. In an illustrative example, a computer may determine based on external research that a course of treatment is not effective, and may in response design a new treatment plan. In a further illustrative example, a computer may determine based on a user's medical history that a course of treatment is not effective, and may in response design a new treatment plan including a new medication, new dosage, or both.

Medication Management: To monitor the use of medication by a patient. A smartphone's webcam or any other wearable device can be partnered with AI to autonomously confirm that patients are taking their medicines and provide a “real-time” measurement to aid patients managing their condition. This feature is particularly useful for people with serious medical conditions, patients who tend to go against doctor advice, and participants in clinical trials. For example, a computer or other suitable machine may be configured to manage dispencing of medicaction, timing of dispence, or other relevant user behaviors/actions, or other aspects of use in connection with a mobile inhaler. In an illustrative example, a computer or other machine may store data regarding a user's activities (e.g., time of use, frequency of use, duration of use, missed doses, extra doses, or unauthorized use), and determine whether the current use habits are effective, suggest new use instructions, or otherwise evaluate the use of the mobile inhaler.

Health Monitoring: Wearable health trackers monitor heart rate, activity levels and measure biomarkers. They can send alerts to the user to inform that a behavioural shift is required, i.e. get more exercise and can share this information to doctors (and AI systems) for additional data points on the needs and habits of patients. For example, a computer or other suitable machine may be configured to track infromation regarding a user's health in connection with a mobile inhaler. In an illustrative example, a computer may be configured to monitor a user's inhalation volume during use of the mobile inhaler (e.g., based on signal from an air flow sensor) to determine the user's breathing capacity. Further, the computer may track the breathing capacity information over time.

In some embodiments, the present disclosure describes the value of artificial intelligence for use in: (1) de-addiction from substance abuse, which result in craving when consumers quit using the addictive compound, and (2) treatment design, (3) medication management and (4) health monitoring of medical treatments. All of which aid in the optimization of diagnosis, treatments, compliance to treatments, and ultimately disease prevention.

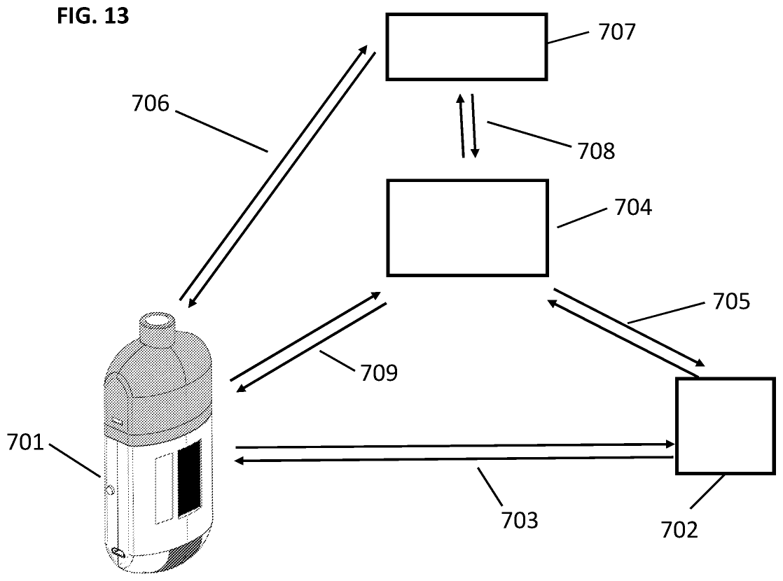

FIG. 13 shows an illustrative ecosystem surrounding a mobile inhaler with artificial intelligence capabilities with a wearable device and the associated dataflows. More specifically, FIG. 13 describes an illustrative system that supports seamless de-addiction from a chemical compound, and in particular nicotine.

In some embodiments, mobile inhaler 701 (including one or more capsules) is coupled wirelessly, via RFID, Internet Protocol, or Bluetooth, for example, to external device 702 (e.g., an external monitoring device). In some such embodiments, external device 702 includes a wearable device, a handheld device, any other similar type of electronic device, or any sutiable combination thereof, that can easily measure a biomarker.

In some embodiments, directed to aiding in nicotine de-addiction, mobile inhaler 701 collects and stores data related to how an individual is using mobile inhaler 701. For example, mobile inhaler 701 may collect data including number of puffs per day (e.g., inhalations per day), duration of puffs, time at which puff takes place, volume of each puff, or other suitable data. In a further example, mobile inhaler 701 measures the nicotine intake that the user draws out of the device by inhalation.

In some embodiments, directed to aiding in nicotine de-addiction, an external device 702 measures specific biomarkers of exposure to nicotine (e.g., cotinine and nicotine), and also measures biomarkers of exposure to conventional smoking (e.g., as carbon monoxide). Following a reading, for example, the external device 702 informs mobile inhaler 701 of measured levels of each biomarker via a wireless communication interface 703. Wireless communication interface 703 allows for “real-time measurements” of nicotine uptake, to be shared with mobile inhaler 701, whereby allowing for the control of nicotine delivery to the user in a gradually decreased manner in order to avoid withdrawal symptoms.

In some embodiments, mobile inhaler 701 includes a touchscreen that displays a visual analog scale user interface and prompts the user to self-evaluate the intensity of craving symptoms.

In some embodiments, mobile inhaler 701 has included in its electronics artificial intelligence capabilities. For example, by using sophisticated algorithms to ‘learn’ features from a large volume of healthcare data (e.g., from many users), and then use the obtained insights to recommend appropriate usage patterns of the device and nicotine levels to the patient for seamless de-addiction.

In some embodiments, mobile inhaler 701 can be equipped with learning and self-correcting abilities to improve its accuracy based on external feedback. In order to achieve these additional learnings, the artificial intelligence system of mobile inhaler 701 is also connected to the internet 707 via a wireless communication system 706. By communicating with the internet, for example, information can be both collected and shared to improved the user's de-addiction protocol by taking in to consideration up-to-date medical information from journals, textbooks and current clinical practices. Further, by communicating with internet 707, the information being generated by the artificial intelligence of mobile inhaler 701 can assist physicians in monitoring and treating users by providing “real-time” measurements of a user's nicotine intake and biomarker levels.

In some embodiments, mobile inhaler 701 and external device 702 are wirelessly connected by respective wireless systems 709 and 705, respectively to an external application 704. This connection provides the capacity to transfer a user's (1) nicotine intake, as measured through the mobile inhaler 701 and (2) nicotine uptake, as measured by the external monitoring to an external application 704, for example a smartphone application. In some embodiments, external application 704 may be coupled to internet 707 via communcations link 708, which may be wired, wireless, optical, or any other suitable link.

The inclusion of artificial intelligence capabilities into mobile inhaler 701 allows users, physicians and any other permitted third-party to: (i) monitor long-term treatment compliance, (ii) collect patient usage patterns for determining successful “step-down” nicotine dosing, and (iii) share the user/patient collected data with external parties for demonstrating treatment. Moreover, the AI system can extract useful information from larger patient populations to assist making real-time inferences and decisions for health risk alert and health outcome prediction. For example, mobile inhaler 701 may be configured to identify trends in usage, liquid selection, or both, to predict usae behavior.

In some embodiments, a blockchain feature is build into the ecosystem surrounding a mobile inhaler. The blockchain is a virtual, public ledger that records everything in a secure and transparent manner. The blockchain allows the free transfer of data through a decentralized environment. All the data is then held in an interlinked network of computers or hand-held devices, owned and operated by the end-users themselves. The blockchaining process allows for at least the following 4 key benefits:

1. Greater transparency. Transaction histories are becoming more transparent through the use of blockchain technology. Because blockchain is a type of distributed ledger, all network participants share the same documentation as opposed to individual copies. That shared version can only be updated through consensus, which means everyone must agree on it. To change a single transaction record would require the alteration of all subsequent records and the collusion of the entire network. Thus, data on a blockchain is more accurate, consistent and transparent than when it is pushed through paper-heavy processes. It is also available to all participants who have permissioned access. To change a single transaction record would require the alteration of all subsequent records and the collusion of the entire network. For example, transactions in connection with a mobile inhaler may be documented with more transparaency through the use of blockchain techniques.

2. Enhanced security. There are several ways blockchain is more secure than other record-keeping systems. Data points must be agreed upon before they are recorded. After a data point is approved, it is encrypted and linked to the previous data point. This, along with the fact that information is stored across a network of computers instead of on a single server, makes it very difficult for “hackers” to compromise the data. Protecting sensitive data is crucial in healthcare—blockchain has an opportunity to really change how critical information is shared by helping to prevent fraud and unauthorized activity. For example, medical records or usage records in connection with a mobile inhaler may be kept secure thorugh the use of blockchain techniques.

3. Improved traceability. Blockchain enables an “audit trail” that shows where a data point came from and every modification that was made on its journey. This historical data can help to verify its authenticity and prevent fraud. For example, data collected in connection with one or mroe mobile inhalers may be more easily traced, authenticated, or otherwise trusted throughthe use of blockchain techniques.

4. Increased efficiency and speed. Any paper-heavy processes is prone to human error and often requires third-party mediation. By streamlining and automating these processes with blockchain, data exchanges can be completed faster and more efficiently. Since record-keeping is performed using a single digital ledger that is shared among participants, there is no need to reconcile multiple ledgers. And when everyone has access to the same information, it becomes easier to trust each other without the need for numerous intermediaries. For example, data interactions, authentications, and transactions may be performed more quickly, efficiently, or both, through the use of blockchain techniques.

In some embodiments, external device 702 is communicatively coupled to communications interface 703 using at least one of RFID, Bluetooth protocol, and an Internet Protocol. In some embodiments, external device 702 includes at least one of a wearable device, a handheld device, a smart phone, and a tablet computer. In some embodiments, processing equipment of mobile inhaler 701, external device 702, or both is configured to determine a dosage of the liquid based at least in part on stored information about the user. In some embodiments, memory of mobile inhaler 701, external device 702, or both is further configured to store information about a plurality of users. In some embodiments, processing equipment of mobile inhaler 701, external device 702, or both is configured to determine a dosage of the liquid based at least in part on stored information about the plurality of users. In some embodiments, processing equipment of mobile inhaler 701, external device 702, or both,
determines the dosage by determining at least one of an amount of the liquid, a duration of atomization, a length of time, and an atomization rate. In some embodiments, processing equipment of mobile inhaler 701, external device 702, or both, includes a communication interface configured to send and receive data from a network (e.g., internet 707). In some embodiments, processing equipment of mobile inhaler 701 is configured to transmit data to external device 702 over a network. In some embodiments, processing equipment of mobile inhaler 701, external device 702, or both, includes a user interface display configured to display data to the user; and
a user input interface configured to receive data inputted by the user. In some embodiments, processing equipment of mobile inhaler 701, external device 702, or both is configured to collect data including a number of inhalations by a user per day, a duration of inhalations by a user, a time at which an inhalation takes place, a volume of an inhalation, any other suitable information, or any combination thereof.

In some embodiments, the liquid of a capsule of mobile inhaler 701 includes nicotine, and processing equipment of mobile inhaler 701, external device 702, or both is configured to collect data comprising intake of the nicotine by the user during inhalation. In some embodiments, external device 702 is configured to measure one or more biomarkers of the user indicative of nicotine exposure. In some embodiments, external device 702 is configured to measure one or more biomarkers of the user indicative of conventional smoking. In some embodiments, external device 702 is configured to communicate to mobile inhaler 701 a measured level of the one or more biomarkers. In some embodiments, processing equipment of mobile inhaler 701, external device 702, or both is configured to control nicotine delivery to the user based at least in part on the one or more biomarkers. In some embodiments, mobile inhaler 701 includes a touchscreen configured to display a visual analog scale user interface, and configured to display prompts the user to self-evaluate the intensity of craving symptoms. The touchscreen is coupled to the processing equipment, and is configured to receive haptic input from the user.

In some embodiments, memory of mobile inhaler 701, external device 702, or both is further configured to store information about a plurality of users, and determine a desired usage pattern of the device based on the stored information about the plurality of users. The usage pattern may include a nicotine level. In some embodiments, memory of mobile inhaler 701, external device 702, or both is further configured to store information about a plurality of users and the communications interface is configured to transmit the stored information about the plurality of users. External device 702 is configured to determine a desired usage pattern (e.g., a nicotine level) of the device based on the stored information about the plurality of users. In some embodiments, at least one of mobile inhaler 701 and the external device 702 is communicatively coupled to a network, and the mobile inhaler system is configured to provide a measurement indicative of the user's use of mobile inhaler 701 to a client coupled to the network. In some embodiments, the measurement includes at least one of a nicotine intake of the user and a biomarker level of the user. In some embodiments, mobile inhaler 701 and external device 702 are communicatively coupled to a client device running an external application. In some embodiments, mobile inhaler 701 is configured to accommodate a capsule configured to store the liquid. The capsule may include an identifier configured to store information about the liquid in the capsule. In some embodiments, at least one of mobile inhaler 701 and external device 702 is configured to store historical information about liquids that have been used in the mobile inhaler.

In some embodiments, mobile inhaler 701 includes a control system configured to manage operation and dispensing of liquid. The control system includes a power source, an electrical port, and control circuitry that is coupled to the power source and the electrical port. The control circuitry is configured to manage power interactions of the power supply, manage charging of the power source via the electrical port, manage data communication with an external device via the electrical port, identify a liquid to be atomized, determine at least one operating parameter, control a piston pump, and control a vibrating mesh membrane. In some embodiments, the operating parameter includes a flow rate of the liquid. In some embodiments, mobile inhaler 701 includes the piston pump having an electrical motor and the control circuitry is configured to control a flow rate of the liquid by controlling a motion of the electric motor. In some embodiments, the control circuitry is configured to control a flow rate of the liquid by controlling a motion of the vibrating mesh membrane. In some embodiments, mobile inhaler 701 includes a pressure sensor coupled to the control circuitry and configured to sense a pressure of the liquid. In some embodiments, the control circuitry is further configured to control at least one of the piston pump and the vibrating mesh membrane based at least in part on the pressure of the liquid. In some embodiments, mobile inhaler 701 includes a pressure sensor coupled to the control circuitry and configured to sense a pressure of air in mobile inhaler 701 (e.g., in the mouthpiece) to detect an inhalation.

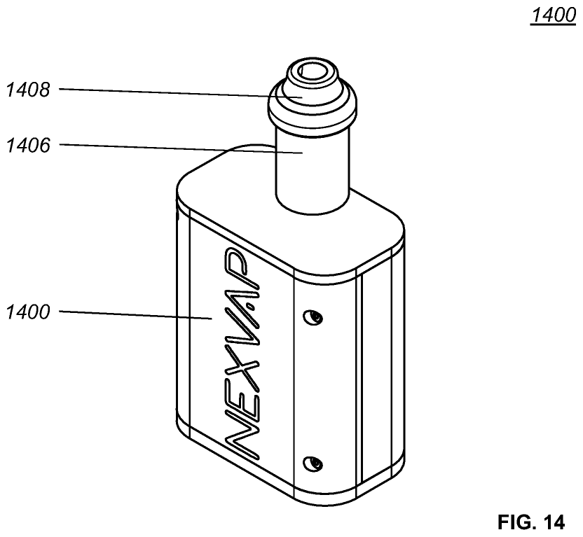

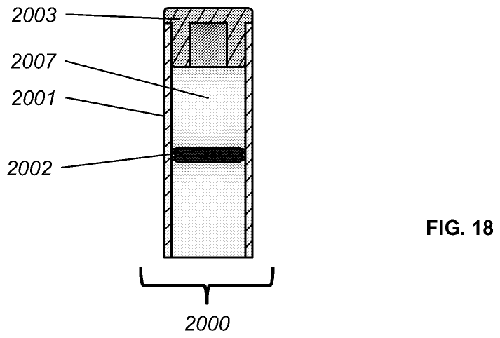

In some embodiments, the present disclsoure is directed to a capsule and nebulization of liquid therein. The capsule and mechansims for pumping the liquid are further described in the context of FIGS. 14-23. For example, FIGS. 14-17 show various views of an illustrative inhaler with a removable front cover, in accordance with some embodiments of the present disclosure. In a further example, FIGS. 18-23 shows various views of a capsule and pumping mechanisms.

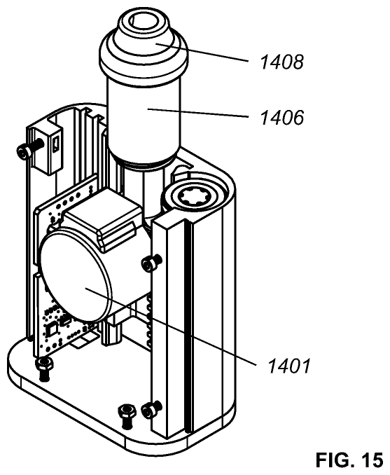

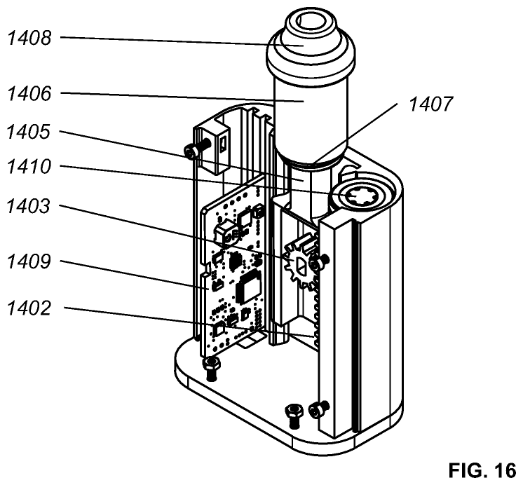

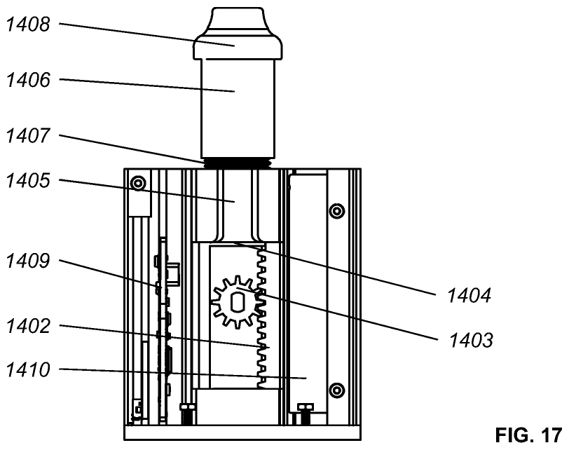

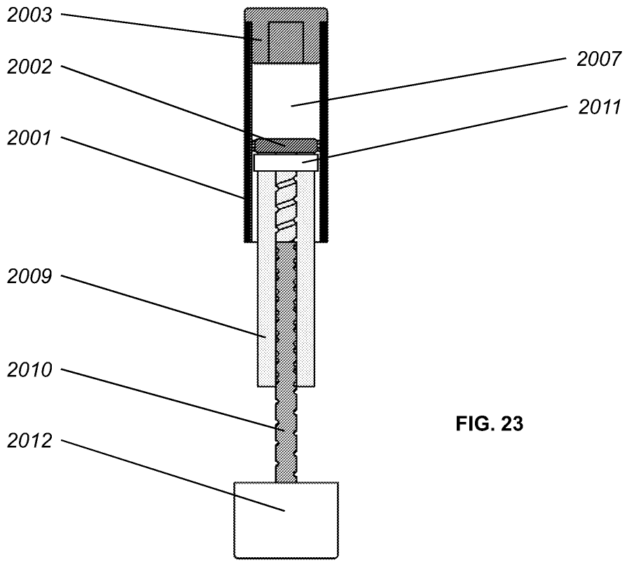

FIG. 14 shows a perspective view of illustrative inhaler 1400 with a removable front cover, in accordance with some embodiments of the present disclosure. For example, inhaler 1400 may be mobile, and configured to be held by a user during use. FIG. 15 shows a perspective view of the inside structure of illustrative inhaler 1400 of FIG. 14 with electric motor 1401, in accordance with some embodiments of the present disclosure. FIG. 16 shows a perspective view of the inside structure of illustrative inhaler 1400 of FIG. 14 without electric motor 1401 to reveal the structural elements behind the electric motor 1401, with a rack 1402 and pinion 1403 transmission mechanism that converts the rotation motion of the shaft of electric motor 101 into linear motion, in accordance with some embodiments of the present disclosure. It will be understood that although illustratively shown in FIGS. 15 and 16 as a rack and pinion, the transmission mechanism may include any suitable mechanism such as, for example, the screw and nut mechanism of FIG. 23. In some embodiments, the configuration and some properties of the electric motor (e.g., position, orientation, speed capability, torque capability, and mounting) may depend on the type of transmission mechanism used. FIG. 17 shows a side view of the inside structure of illustrative inhaler 1400 of FIG. 14 with rack 1402 and pinion 1403 transmission mechanism that converts the rotation motion of the shaft of electric motor 1401 into linear motion, without the electric motor 1401 to reveal the structural elements behind the electric motor 1401, in accordance with some embodiments of the present disclosure. For example, as illustrated in FIG. 17, as pinion 1403 rotates about its center, rack 1402 may move up or down depending on the direction of rotation, thus moving piston 1405 up and down accordingly.

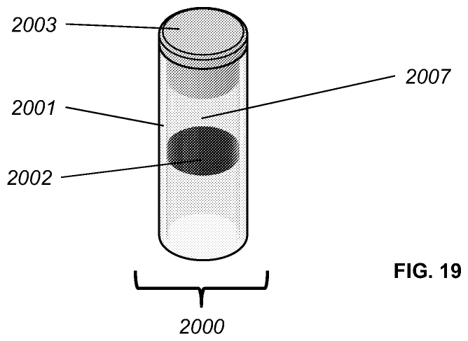

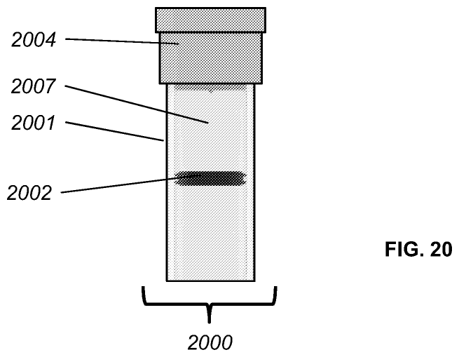

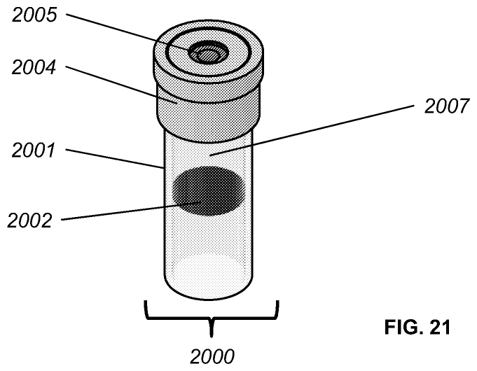

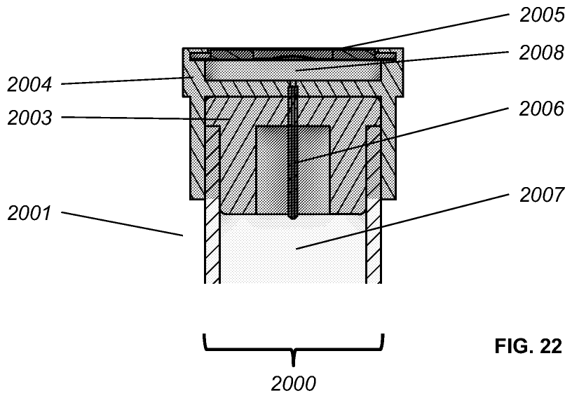

FIG. 18 shows a cross-sectional view of capsule 2000, in accordance with some embodiments of the present disclosure. For example, capsule 2000 may be cylindrical, and the center axis may be oriented vertically in FIG. 18. Accordingly, sliding gasket 2002 may be configured to translate primarily along the center axis. FIG. 19 shows a perspective view of capsule 2000 of FIG. 18, in accordance with some embodiments of the present disclosure. In some embodiments, for example, capsule body 2001 is of cylindrical shape and is sealed on its front end by sealing cap 2003, which is hollow in its center. Sliding gasket 1407 or 2002 seals liquid 2007 at the back end of capsule 2000 and ensures that liquid 2007 is not in contact with air and that capsule 2000 containing liquid 2007 is air-free and air-tight. FIG. 20 shows a side view of capsule 2000 of FIG. 18, fitted into a membrane holder 2004, in accordance with some embodiments of the present disclosure. FIG. 21 shows a perspective view of capsule 2000 fitted into membrane holder 2004, in accordance with some embodiments of the present disclosure. FIG. 22 shows a cross-sectional view of membrane holder 2004 mounted on the front end of capsule 2000, in accordance with some embodiments of the present disclosure. Capsule 2000 is configured to be arranged in capsule holder 1406, and is engaged by the rack and pinion mechanism to allow pumping of liquid 2007 of capsule 2000. Capsule 2000 may be the same as liquid container 104 of FIG. 1 and FIG. 8, for example.

In some embodiments, capsule body 2001 is of cylindrical shape and is sealed on its front end by sealing cap 2003, which is hollow in its center. Sliding gasket 1407 or 2002 seals liquid 2007 at the back end of capsule 2000 and ensures that liquid 2007 is not in contact with air and that capsule 2000, containing liquid 2007, is air-free and air-tight. In some embodiments, the front end of capsule 2000 is fitted into membrane holder 2004 where the piezoelectric driven vibrating mesh membrane 2005 is located. Vibrating mesh membrane 2005 is mounted on the upper side of membrane holder 2004, which is connected to capsule body 2001.

In some embodiments, when the front end of capsule body 2001 is inserted in membrane holder 2004, needle 2006 perforates sealing cap 2003. Liquid 2007 is drawn through needle 2006 to the membrane chamber 2008 which is under vibrating mesh membrane 2005. Sliding gasket 1407 or 2002 is pushed upwards by rack 1402, thereby forcing liquid 2007 through needle 2006 and into membrane chamber 2008. The pumped liquid of liquid 2007 is therefore in contact with vibrating mesh membrane 2005, which is configured to nebulize the liquid into an aerosol driven by an electric signal coming from electronic board 1409.

In some embodiments, the inhaler contains electric motor 1401 coupled to a transmission gear mechanism that pushes sliding gasket 1407 (e.g., a slidable seal) contained in a disposable capsule 2000, which contains liquid 2007 to be nebulized. Capsule 2000 includes sealing cap 2003 that is perforated by needle 2006, and which allows liquid 2007 to be drawn to vibrating mesh membrane 2005 for nebulization into an aerosol.

In some embodiments, electric motor 1401 may include a DC motor of the brushed or brushless type, a stepper motor, or any other suitable type of electric motor. In some embodiments, electric motor 1401 includes a stepper motor configured to control the frequency of steps. In some such embodiments, electronic board 1409 can precisely control the number of turns or fractions of turns made by the motor shaft, or pinion 1403, in any given time interval. In an illustrative example, when a DC motor is used, electronic board 1409 can control the rotating speed of electric motor 1401 by means of current or voltage control or any other means, therefore controlling the number of turns (i.e., rotations), or fraction of turns, of pinion 1403 in any given time interval.

The transmission mechanism that converts the rotating motion of electric motor 1401 or 2012 into linear motion (e.g., to linearly move sliding gasket 1407), can be of a rack and pinion type (e.g., rack 1402 and pinion 1403), or of a screw and nut type (e.g., screw 2010 and nut 2009), any other suitable equivalent mechanism, or any combination thereof.

In some embodiments, pinion 1403 is engaged with rack 1402, which is configured to slide according to the rotations or fraction of rotations of pinion 1403. On the top end of rack 1402 is affixed piston 1405 or 2011 (e.g., via welding, fastening, adhering, or piston 1405 and rack 1402 may be a single machined component). Piston 1405 or 2011 slides inside the back end of capsule body 2001 and pushes sliding gasket 1407 or 2002 inside the back end of capsule 2000, according to the rotation of electric motor 1401 or 2012. This allows a desired volume of liquid 2007 (e.g., a metered dose) to be displaced from capsule 2000 towards vibrating mesh membrane 2005. As piston 1405 and sliding gasket 1407 move linearly, functioning as a piston pump, the volume of liquid in capsule 2000 may change accordingly (e.g., liquid may be pumped from capsule 2000).

In some embodiments, nut 2009 is affixed to piston 1405 or 2011 (e.g., via welding or other securement) and pushes sliding gasket 1407 or 2002 inside the back end of the capsule 2000, according to the rotation of electric motor 1401 or 2012. This allows a desired volume of liquid 2007 (e.g., a metered dose) to be displaced from capsule 2000 towards the vibrating mesh membrane (not shown in FIGS. 18-23).

In some embodiments, the present disclosure is directed to an electric motor 1401 or 2012 combined with a rack and pinion mechanism (e.g., rack 1402 and pinion 1403) that pushes sliding gasket 1407 or 2002 contained in disposable capsule 2000 that contains liquid 2007 to be nebulized. The capsule 2000 has a sealing cap 2003 that is perforated by needle 2006, which allows liquid 2007 to be drawn to mesh membrane 2005 for nebulization into an aerosol.

In some embodiments, electric motor 1401 or electric motor 2012 may include a reduction gear for the achievement of optimum torque and rotation speed of the pinion 1403. The motion of electric motor 1401 or electric motor2012 is controlled by electronic board 1409, that determines the number of steps to be performed by the motor in any given time unit. By controlling the frequency of the steps, electronic board 1409 can precisely control the number of turns or fractions of turns made by pinion 1403 in any given time interval. In some embodiments, electric motor 1401, electric motor 2012, or both, includes a brush-type DC motor, a brushless-type DC motor, a stepper motor, or any other suitable type of electric motor. In an illustrative example, wherein electric motor 1401 includes a stepper motor, by controlling the frequency of steps, electronic board 1409 can precisely control the number of turns or fractions of turns made by the motor shaft, or pinion 1403, in any given time interval. In some embodiments, when a DC motor is included, electronic board 1409 is configured to control the rotating speed of electric motor 1401 or electric motor 2012 by means of current or voltage control or any other means, therefore controlling the number of turns, or fraction of turns, of pinion 1403 in any given time interval.

In some embodiments, the transmission mechanism that converts the rotating motion of electric motor 1401 or electric motor 2012 into linear motion, can include a rack and pinion type mechanism (e.g., rack 1402 and pinion 1403), a screw type mechanism (e.g., screw 2010 and nut 2009), or any other suitable equivalent mechanism.

In some embodiments, pinion 1403 is engaged with rack 1402, which is configured to slide according to the rotations of pinion 1403. One end of rack 1402 is coupled to piston 105 or 2011 that pushes sliding gasket 1407 or 2002 inside the back end of capsule 2000. Piston 1405 or 2011 slides inside the back end of capsule 2000 when rack 1402 slides according to the rotation of pinion 1403. Piston 1405 or piston 2011 is in contact with respective gasket 1407 or gasket 2002 contained in capsule 2000. Gasket 1407, or gasket 2002, ensures hermeticity (e.g., such that no liquid from capsule 2000 leaks out and air does not leak in), even when sliding inside capsule 2000. The linear distance travelled by piston 1405 or piston 2011 inside capsule 2000 is controlled by the number of rotational steps performed by respective electric motor 1401 or electric 2012, as controlled by electronic board 1409. This position control allows the inhaler to meter a dose of a desired volume of liquid 2007 to be displaced from capsule 2000 towards vibrating mesh membrane 2005.

In some embodiments, the nut 2009 is welded to a piston 1405 or piston 2011 and pushes the respective sliding gasket 1407 or 2002 inside the back end of the capsule 2000, according to the rotation of the respective electric motor 1401 or 2012. This position control allows the inhaler to meter a dose of a desired volume of liquid 2007 to be displaced from capsule 2000 towards vibrating mesh membrane 2005 (not shown).

In some embodiments, the front end of the capsule 2000 is coupled to vibrating mesh membrane 2005 by membrane holder 2004, where vibrating mesh membrane 2005 is mounted. Capsule 2000 is coupled to the membrane chamber 2008 through needle 2006, allowing liquid 2007 to flow from capsule 2000 into membrane chamber 2008 without leakage (e.g., of liquid out or air in). Vibrating mesh membrane 2005 is mounted on the upper side of membrane chamber 2008, within membrane holder 2004. Membrane holder 2004 is contained in mouthpiece 1408 in such a way that it vibrates freely at its optimum rate (e.g., frequency) while ensuring full hermeticity. In some embodiments, mouthpiece 1408 and capsule holder 1406 may be integrated as a single piece or component, and accordingly may be referred to as a mouthpiece, a capsule holder, or either.

In some embodiments, vibrating mesh membrane 2005 is configured to nebulize liquid 2007 contained in membrane chamber 2008 at a rate that is based at least in part on the number and size of the holes of the membrane itself, and the frequency, amplitude, or duty cycle of the controlling signal, generated by electronic board 1409.

In some embodiments, membrane chamber 2008 may include a pressure sensor (not shown in FIGS. 18-23), that is configured to measure the pressure of liquid 2007 inside membrane chamber 2008. The corresponding measure of pressure is fed to electronic board 1409. For example, in some embodiments, the pressure sensor is powered by electronic board 1409 (e.g., to provide electronic excitation for the output signal). In some embodiments, the pressure sensor outputs a signal (e.g., a voltage, current, impedance, or waveform) to electronic board 1409.

In some embodiments, the inhaler may include a pressure on-off sensor (not shown), that is configured to detect when the user inhales through mouthpiece 1408 of the inhaler and feeds the detection signal to electronic board 1409. In an illustrative example, the on-off pressure sensor may include a pressure switch, configured to open or close an electrical circuit based on a pressure threshold. In some embodiments, a push button may be included on the outer surface of the inhaler. The push button may be configured to be operated by the user, and may be configured to start operation of the inhaler. For example, the push button may be electrically coupled to electronic board 1409, which may be configured to detect a position (e.g., a throw) of the push button (e.g., on, off).

In some embodiments, the inhaler includes battery 1410 that supplies electric power (e.g., voltage and current) to circuits in the inhaler. In some such embodiments, the inhaler includes a USB connector (not shown) for the charging of battery 1410 and for communications of data between electronic board 1409 and any other external handheld devices such as, for example, a monitor, handheld smartphone or other suitable device.

In some embodiments, electronic board 1409 includes a microcontroller, that is configured to manage the operation of the inhaler. Electronic board 1409 may include any suitable control circuitry configured to control any suitable aspect of operation of the inhaler. It will be understood that electronic board 1409 is illustrative, and that any suitable configuration of one or more electronic boards (e.g., printed circuit boards (PCBs)) may be included. For example, electronic board 1409 may include several PCBs and other components, electrically coupled by pins, cables, harnesses, connectors, any other suitable electrical coupling, or any combination thereof. In a further example, electronic board 1409 may include a single board having a desired functionality (e.g., piezoelectric control of a vibrating mesh membrane, motor control, sensor interface, power management circuitry, or a combination thereof). In some embodiments, electronics board 1409 need not include a PCB, and may, for example, include wired circuits coupled together. Any suitable control circuitry may be used in accordance with the present disclosure.

In some embodiments, for example, electronic board 1409 is activated by the user through response of the on-off pressure sensor to the user's inhalation, or by the user pushing the push button. In response, electronic board 1409 controls the frequency of the steps of electric motor 1401 or 2012, and the frequency, amplitude and duty cycle of the controlling signal for vibrating mesh membrane 2005, in such a way that the flow rate of liquid 2007 out of capsule 2000 matches the nebulization rate of vibrating mesh membrane 2005. For example, electronic board 1409 may determine a desired flow rate of liquid 2007, and accordingly may control the piston pump (e.g., motion of piston 1405 and sliding gasket 1407 as controlled by motor 1401) and the nebulizer (e.g., via vibrating mesh membrane 2005) such that both achieve this desired flow rate.

In some embodiments, electronic board 1409 may use a measurement of pressure of liquid 2007 inside membrane chamber 2008 to adjust control signals to electric motor 1401 or 2012 and to vibrating mesh membrane 2005. For example, electronic board may be configured to adjust the control signals in such a way that the pressure of the liquid remains within a suitable pre-determined range of pressure values, allowing for the optimal nebulization of liquid 2007 through vibrating mesh membrane 2005.

In some embodiments, in which electronic board 1409 includes the microcontroller, the microcontroller is configured to keep track of the steps made by electric motor 1401 or 2012. Accordingly, electronic board 1409 can precisely meter the amount of liquid 2007 (e.g., a dose) that has been nebulized through vibrating mesh membrane 2005 at any given moment. For example, electronic board 1409 may be configured to precisely meter an instantaneous flow rate, an effective flow rate (e.g., a low-pass filtered flow rate in view of fluctuations, or an average flow rate), a cumulative volume of liquid, or a combination thereof.

In some embodiments, the size and shape of the inhaler is such that it can easily be hand held. In some embodiments, the inhaler is hermetic and leak-proof under all atmospheric conditions or any given angle at which it is held. In some embodiments, the shape and structure of the inhaler is arranged in such a way that capsule 2000 can be changed at any moment (e.g., swapped with a different capsule, containing the same liquid or a different liquid), without any leakage of liquid 2007 from capsule 2000 or from the inhaler. Electronic board 1409 detects the insertion of a new capsule and recognizes the specific liquid content of the new capsule (e.g., which may be the same as liquid 2007 of capsule 2000). For example, this information is used for the computation of the appropriate quantity of liquid to be nebulized by electronic board 1409. For example, capsule 2000 may include an identifier such as an RFID tag, a bar code, or other suitable machine-readable identifier. In a further example, the user may input identification information using a user interface (e.g., or from an external user device via the USB data connection).

In some embodiments, battery 1410 is charged through a USB connector and supplies the voltage to a voltage regulator (not shown). The voltage regulator supplies a regulated voltage to the microcontroller (not shown) located on electronic board 1409, an electric motor driver (e.g., not shown but optionally included in electronic board 1409), a membrane driver (e.g., not shown but optionally included in electronic board 1409), and to an optional display (e.g., not shown but optionally included as part of the inhaler).

In some embodiments, the USB connector can also be used as a communication port for the data transfer between the microcontroller and an external device, allowing for the management and the programming of the microcontroller characteristics.

In an illustrative example, the inhaler includes an electric motor 1401 or 2012 connected to a pinion 1403, which is connected to a reduction gear inside the electric motor 1401 or 2012. The pinion 1403 is connected to a rack 1402, driving a piston 1405 or 2011 inside the capsule holder 1406 where the capsule 2000 is inserted. In a further example, the inhaler includes a piston 1405 or piston 2011 with a plastic or rubber gasket 1407 or 2002 slides inside the capsule 2000, the capsule 2000 is inside the capsule holder 1406. In a further example, the inhaler includes a piezoelectric driven mesh membrane 2005 is in the mouthpiece 1408. The mouthpiece 1408 sits on top of the capsule holder 1406 which contains the capsule 2000. In a further example, the inhaler includes an electronic board 1409 controls the electric motor 1401 or 2012, and the piezoelectric driven mesh membrane 2005 in the mouthpiece 1408. The electronic board 1409 is connected to a USB connector not shown and to an optional display (not shown). In a further example, the inhaler includes a rechargeable battery 1410 charged from a charging circuit of any type located on the electronic board 1409. In a further example, the inhaler includes an on-off pressure sensor (not shown) that activates the electronic board 1409 when a user inhales through the mouthpiece 1408, is located in the mouthpiece 1408, or in any other location inside the inhaler. In a further example, the inhaler includes a push button (not shown) can be located in any position within the inhaler that activates the electronic board 1409.

FIG. 23 shows a cross-sectional view of capsule body 2001 with its sealing cap 2003, containing a liquid 2007 sealed by a sliding gasket 1407 or 2002. The drawing further represents the transmission mechanism that converts the rotating motion of the electric motor 1401 or 2012 into linear motion, through a screw 2010 and nut 2009 type, in accordance with some embodiments of the present disclosure. As illustrated in FIG. 23, capsule body 2001 includes sealing cap (i.e., cap 2003), which which seals liquid 2007 along with sliding gasket 2002 (e.g., or sliding gasket 1407 of inhaler 1400). FIG. 23 further illustrates the transmission mechanism that is configured to convert the rotating motion of electric motor 2012 into linear motion, through a screw and nut type mechanism (e.g., screw 2010 and nut 2009).

In some embodiments, a mobile inhaler (e.g., mobile inhaler 1400) includes a capsule comprising a liquid and a slidable seal (e.g., gasket 1407 or 2002), a piston pump configured to pump the liquid, a vibrating mesh membrane coupled to the capsule and configured to atomize the liquid, and control circuitry electrically coupled to the electric motor and the vibrating mesh membrane. The piston pump includes an electric motor having a shaft, a transmission mechanism coupled to the shaft, and a piston coupled to the transmission mechanism and arranged at one end of the capsule. The piston is configured to apply force to the slidable seal. The control circuitry is configured to control a rotation of the electric motor, and control an actuation of the vibrating mesh membrane. In som embodiments, the transmission mechanism comprises a rack and pinion mechanism. In some embodiments, the pinion (e.g., pinion 1403) is rigidly coupled to the shaft and the rack (e.g., rack 1402) is rigidly coupled to the piston (e.g., piston 1405 or 2011). In some embodiments, the transmission mechanism includes a screw (e.g., screw 2010) and nut (e.g., nut 2009) mechanism. In some embodiments, the screw is rigidly coupled to the shaft and the nut is rigidly coupled to the piston. In soem embodiments, the mobile inhaler includes a mouthpiece (e.g., mouthpiece 1408) configured to be inhaled from by a user. In some embodiments, the capsule (e.g., capsule 2000) is arranged at least partially within the mouthpiece (e.g., mouthpiece 1408). In some embodiments, the capsule (e.g., capsule 2000) is arranged completely within the mouthpiece (e.g., mouthpiece 1408). In some embodiments, the electric motor (e.g., electric motor 1401 or electric motor 2012) includes a stepper motor, and the control circuitry includes a stepper motor controller. In some embodiments, the electric motor (e.g., electric motor 1401 or electric motor 2012) includes a DC motor, and the control circuitry comprises a DC motor controller. In some embodiments, the control circuitry (e.g., electronic board 1409) is configured to determine a flow rate of the liquid based at least in part on a rotation of the shaft of the electric motor (e.g., electric motor 1401 or electric motor 2012). In some embodiments, the control circuitry (e.g., electronic board 1409) is configured to determine a flow rate of the liquid based at least in part on an atomization rate of the liquid by the vibrating mesh membrane. In some embodiments, the control circuitry (e.g., electronic board 1409) is configured to identify the liquid (e.g., of capsule 2000). In soem embodiments, the capsule (e.g., capsule 2000) includes an identifier, and the control circuitry is further configured to detect the identifier, and identify the liquid based at least in part on the identifier. In some embodiments, the control circuitry is further configured to receive user input, and identify the liquid based at least in part on the user input. In some embodiments, the control circuitry (e.g., electronic board 1409) is further configured to determine a desired flow rate of the liquid based at least in part on identifying the liquid. In some embodiments, the mobile inhaler (e.g., mobile inhaler 1400) includes a membrane holder configured to engage the capsule. For exampel, the capsule may include a sealing cap, the vibrating mesh membrane is arranged in the membrane holder, the membrane holder includes a membrane chamber adjacent to the vibrating mesh membrane, and the membrane holder includes a needle coupled to the membrane chamber and configured to pierce the sealing cap thereby allowing the liquid to flow from the capsule, through the needle, and to the membrane chamber. In soem embodiments, a power source is coupled to the control circuitry. In some embodiments, the mobile inhaler (e.g., mobile inhaler 1400) includes a port configured to transmit at least one of electrical power and data to and from an external device. In some embodiments, the mobile inhaler (e.g., mobile inhaler 1400) includes a pressure sensor electrically coupled to the control circuitry and configured to sense a pressure of the liquid in at least one of the capsule and the membrane chamber. In some embodiments, the control circuitry (e.g., electronic board 1409) is configured to control at least one of the rotation of the electric motor and the actuation of the vibrating mesh membrane based at least in part on the pressure of the liquid. In some embodiments, the mobile inhaler (e.g., mobile inhaler 1400) includes a pressure sensor electrically coupled to the control circuitry and configured to sense an inhalation of the user by sensing an air pressure in the inhaler.

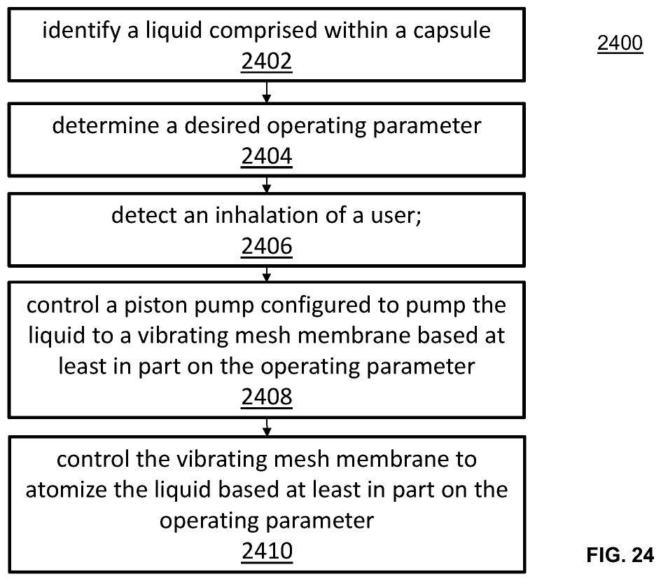

FIG. 24 is a flowchart of illustrative process 2400 for controlling a mobile inhaler, in accordance with some embodiments of the present disclosure.

Step 2402 includes processing equipment identifying a liquid comprised within a capsule. For example, the processing equipment may be configured to identify an indentifer of the capsule (e.g., as described in the context of FIG. 13).

Step 2404 includes processing equipment determining a desired operating parameter. The desired operating parameter may include a liquid flow rate, total liquid volume, an atomization rate, a vibration frequency (e.g., of a vibrating mesh membrane), any other suitable operating parameter, or any combination thereof.

Step 2406 includes processing equipment detecting an inhalation of a user. In some embodiments, inhalation is detected based on input from an air flow sensor (e.g., air flow sensors 111, air flow sensor 303, air flow sensor 122, air flow sensor 312, or any other suitable sensor) for monitoring the air flow into the mouthpiece. In some embodiments, inhalation may be detected by a pressure sensor (e.g., a relatively low pressure is detected in the mouthpiece or body indicating an inhalation).

Step 2408 includes processing equipment controlling a pump configured to pump the liquid to a vibrating mesh membrane based at least in part on the operating parameter. In some embodiments, step 2408 is perfomed in response to the detecting of step 2406. The pump may include a piston pump and, for example, any of the mechanisms described in the context of FIGS. 14-17, and 23. In some embodiments, the pump may incude, for example, a micro-pump described in the context of FIGS. 1-6.

Step 2410 includes processing equipment controlling the vibrating mesh membrane to atomize the liquid based at least in part on the operating parameter. In some embodiments, step 2410 is perfomed in response to the detecting of step 2406. The vibrating mesh membrane may include, for example, vibrating mesh membrane 108 described in the context of FIGS. 1-6.

In some embodiments, process 2400 includes the processing equipment an operating mode (e.g., any of the illustrative operation modes of FIGS. 11-12).

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed is:

1. A mobile inhaler comprising:
a mouthpiece comprising:
one or more air ducts configured for intaking air; and
an aperture configured to interface to a user and deliver aerosol to the user during inhalation
a body coupled to the mouthpiece;
a replaceable capsule configured to contain a liquid, and removably mounted in the body;
a power source configured to provide electrical power;
processing equipment coupled to the power source;
a liquid pump system configured for pumping the liquid; and
a vibrating mesh atomizer configured for atomizing the liquid, wherein the liquid pump system is further configured to pump the liquid to the atomizer.

2. The mobile inhaler of claim 1, wherein the atomizer comprises:
a vibrating mesh membrane comprising a plurality of holes;
a piezoelectric actuator coupled to the vibrating mesh membrane, wherein the processing equipment is configured to actuate the piezoelectric actuator; and
a liquid membrane chamber configured to hold the liquid in contact with the vibrating mesh membrane.

3. The mobile inhaler of claim 1, wherein the liquid pump system comprises a micro-pump configured to pump the liquid from the capsule to the atomizer.

4. The mobile inhaler of claim 1, wherein the processing equipment comprises:
a USB connector;
a first air flow sensor;
a second air flow sensor
at least one switch coupled to the first air flow sensor;
an input detector configured to detect a first electrical characteristic of the DC power;
a power converting unit configured to convert a DC power to AC power for actuating the atomizer;
an output detector configured to detect a second electrical characteristic of the AC power;
a voltage regulator configured to regulate a voltage of the power source;
a micro-pump driving unit configured to control the micro-pump, wherein the power source provides power to the micro-pump driving unit; and
a microcontroller unit configured to manage the voltage regulator, the micro-pump driving unit, and the power converting unit, and configured to receive the first and second electrical characteristics.

5. The mobile inhaler of claim 4, wherein the power converting unit comprises:
a DC-DC converter configured to convert a first DC signal to a second DC signal;
a DC-AC converter configured to generate a first AC signal from the second DC signal; and
a transformer configured to receive the first AC signal and provide a second AC signal to the piezoelectric actuator.

6. The mobile inhaler of claim 4, wherein the processing equipment further comprises a second air flow sensor configured to sense an airflow and provide a corresponding signal to the microcontroller unit.

7. The mobile inhaler of claim 4, wherein the processing equipment further comprises a personal identification recognition means.

8. The mobile inhaler of claim 4, wherein the processing equipment further comprises a user interface display configured to display data to the user.

9. The mobile inhaler of claim 1, wherein the capsule further comprises:
a capsule body;
a deformable balloon arranged within the capsule body, wherein the deformable balloon is liquid tight, and wherein there is an inner volume between at least a portion of the deformable balloon and the capsule body; and
wherein the bottom aperture is configured to pass the capsule through during installation and removal.

10. The mobile inhaler of claim 9, wherein the capsule further comprises:
a front seal configured to seal the deformable balloon from outside of the capsule body;
a front plug configured to rigidly hold the front seal to the capsule body; and
a rear plug configured to seal the inner volume from outside of the capsule body.

11. The mobile inhaler of claim 10, wherein the rear plug comprises a one-way valve coupling the inner volume to outside of the capsule body.

12. The mobile inhaler of claim 1, wherein the capsule further comprises identification means configured to store information about the liquid in the capsule.

13. The mobile inhaler of claim 1, wherein the liquid comprises:
0.5-20% of total volume Nicotine concentration, wherein the total Nicotine concentration comprises:
x freebase Nicotine at a pH of 8.5 to 9.5, wherein x ranges from 0-100%, and
100%-x Nicotine salts at a pH of 4 to 5;
0.5-25% of total volume Aromatic compounds dissolved in propylene glycol;
20-90% of total volume distilled water concentration; and
2-10% of total volume vanillin dissolved in propylene glycol.

14. The mobile inhaler of claim 1, further comprising a needle configured to puncture the capsule, wherein the needle is coupled to the liquid pump system to deliver the liquid from the capsule.

15. The mobile inhaler of claim 1, wherein the vibrating mesh atomizer comprises:
a vibrating mesh membrane comprising a plurality of holes;
a piezoelectric actuator coupled to the vibrating mesh membrane, wherein the processing equipment is configured to actuate the piezoelectric actuator; and
a vibrating mesh membrane holder configured to house the vibrating mesh membrane and a liquid membrane chamber configured to hold the liquid in contact with the vibrating mesh membrane.

16. The mobile inhaler of claim 15, wherein the vibrating mesh holder comprises:
an opening; and
a groove arranged in the opening, wherein the vibrating mesh membrane and the piezoelectric actuator are arranged in the groove, and wherein the groove comprises a thickness substantially equal to a thickness of the vibrating mesh membrane.

17. The mobile inhaler of claim 16 wherein the groove and the vibrating mesh membrane are hermetically sealed to each other.

18. The mobile inhaler of claim 15, wherein the vibrating mesh membrane and the vibrating mesh membrane holder are adhered to each other.

19. The mobile inhaler of claim 15, wherein the vibrating mesh membrane is rigidly affixed along an outer perimeter to the vibrating mesh membrane holder, and wherein a preload force on the vibrating mesh membrane is negligible.

20. The mobile inhaler of claim 15, wherein the vibrating mesh membrane does not significantly plastically deform.

21. The mobile inhaler of claim 15, wherein the vibrating mesh membrane holder is formed using a technique selected from Fused Deposition Modelling (FDM), Fused Filament Fabrication (FFF), and three-dimensional (3D) printing.

22. The mobile inhaler of claim 15, wherein the vibrating mesh membrane holder is comprised of a material selected from a Thermo Plastic Elastomer (TPE), Thermo Plastic Polyurethane (TPU), Acrylonitrile Butadiene Styrene (ABS), Poly Lactic Acid (PLA), and a polymerized resin.

23. The mobile inhaler of claim 15, wherein a flowrate of the liquid in the liquid pump system is matched to an atomization rate of the liquid by the vibrating mesh atomizer.

24. The mobile inhaler of claim 15, further comprising a pressure sensor configured to sense a pressure of the liquid in the vibrating mesh membrane holder, wherein the pressure sensor is coupled to the processing equipment.

25. The mobile inhaler of claim 15, wherein the vibrating mesh holder comprises a chamber comprising one or more walls and a bottom floor, wherein the bottom floor comprises a hole coupled to the liquid pump system.

26. The mobile inhaler of claim 25, wherein the one or more walls comprise a flexible wall configured to limit a pressure fluctuation of the liquid in the vibrating mesh membrane holder, and wherein the flexible wall allows a volume of the liquid in the vibrating mesh membrane holder to change in response to the pressure.

27. The mobile inhaler of claim 26, further comprising a pressure sensor configured to sense a pressure of the liquid, and wherein the processing equipment is configured to identify a change of the volume of the liquid in the vibrating mesh membrane holder based on a signal from the pressure sensor.

28. The mobile inhaler of claim 26, further comprising a pressure switch configured to open and close a circuit based on a deformation of the flexible wall, wherein the pressure switch is coupled to the processing equipment.

29. A mobile inhaler configured to atomize a liquid for inhalation, the mobile inhaler comprising:
processing equipment;
a liquid pump system coupled to the processing equipment and configured for pumping the liquid; and
a vibrating mesh atomizer configured for atomizing the liquid, wherein the liquid pump system is further configured to pump the liquid to the atomizer, wherein the vibrating mesh atomizer comprises:
a vibrating mesh membrane comprising a plurality of holes,
a piezoelectric actuator coupled to the vibrating mesh membrane, wherein the processing equipment is configured to actuate the piezoelectric actuator, and
a vibrating mesh membrane holder configured to house the vibrating mesh membrane and a liquid membrane chamber configured to hold the liquid in contact with the vibrating mesh membrane.

30. The mobile inhaler of claim 29, wherein the vibrating mesh holder comprises:
an opening; and
a groove arranged in the opening, wherein the vibrating mesh membrane and the piezoelectric actuator are arranged in the groove, and wherein the groove comprises a thickness substantially equal to a thickness of the vibrating mesh membrane.

31. The mobile inhaler of claim 30, wherein the groove and the vibrating mesh membrane are hermetically sealed to each other.

32. The mobile inhaler of claim 29, wherein the vibrating mesh membrane and the vibrating mesh membrane holder are adhered to each other.

33. The mobile inhaler of claim 29, wherein the vibrating mesh membrane is rigidly affixed along an outer perimeter to the vibrating mesh membrane holder, and wherein a preload force on the vibrating mesh membrane is negligible.

34. The mobile inhaler of claim 29, wherein the vibrating mesh membrane does not significantly plastically deform.

35. The mobile inhaler of claim 29, wherein the vibrating mesh membrane holder is formed using a technique selected from Fused Deposition Modelling (FDM), Fused Filament Fabrication (FFF), and 3D printing.

36. The mobile inhaler of claim 29, wherein the vibrating mesh membrane holder is comprised of a material selected from a Thermo Plastic Elastomer (TPE), Thermo Plastic Polyurethane (TPU), Acrylonitrile Butadiene Styrene (ABS), Poly Lactic Acid (PLA), and a polymerized resin.

37. The mobile inhaler of claim 29, wherein a flowrate of the liquid in the liquid pump system is matched to an atomization rate of the liquid by the vibrating mesh atomizer.

38. The mobile inhaler of claim 29, further comprising a pressure sensor configured to sense a pressure of the liquid in the vibrating mesh membrane holder, wherein the pressure sensor is coupled to the processing equipment.

39. The mobile inhaler of claim 29, wherein the vibrating mesh holder comprises a chamber comprising one or more walls and a bottom floor, wherein the bottom floor comprises a hole coupled to the liquid pump system.

40. The mobile inhaler of claim 29, wherein the one or more walls comprise a flexible wall configured to limit a pressure fluctuation of the liquid in the vibrating mesh membrane holder, and wherein the flexible wall allows a volume of the liquid in the vibrating mesh membrane holder to change in response to the pressure.

41. The mobile inhaler of claim 40, further comprising a pressure sensor configured to sense a pressure of the liquid, and wherein the processing equipment is configured to identify a change of the volume of the liquid in the vibrating mesh membrane holder based on a signal from the pressure sensor.

42. The mobile inhaler of claim 40, further comprising a pressure switch configured to open and close a circuit based on a deformation of the flexible wall, wherein the pressure switch is coupled to the processing equipment.

43. A mobile inhaler comprising:
a mouthpiece comprising:
one or more air ducts configured for intaking air; and
an aperture configured to interface to a user and deliver aerosol to the user during inhalation
a body coupled to the mouthpiece;
a replaceable capsule configured to contain a liquid, and removably mounted in the body;
a power source configured to provide electrical power;
processing equipment coupled to the power source, wherein the processing equipment comprises memory configured to store information about the user;
a liquid pump system configured for pumping the liquid; and
a vibrating mesh atomizer configured for atomizing the liquid, wherein the liquid pump system is further configured to pump the liquid to the atomizer.

44. The mobile inhaler of claim 43, wherein the processing equipment is configured to determine a dosage of the liquid based at least in part on stored information about the user.

45. The mobile inhaler of claim 43, wherein the memory is further configured to store information about a plurality of users.

46. The mobile inhaler of claim 45, wherein the processing equipment is configured to determine a dosage of the liquid based at least in part on stored information about the plurality of users.

47. The mobile inhaler of any of claims 44 and 46, wherein determining the dosage comprises determining at least one of an amount of the liquid, a duration of atomization, a length of time, and an atomization rate.

48. The mobile inhaler of claim 43, wherein the processing equipment further comprises a communication interface configured to send and receive data from a network.

49. The mobile inhaler of claim 48, wherein the processing equipment is configured to transmit data to an external device over the network.

50. The mobile inhaler of claim 43, wherein the processing equipment further comprises:
a user interface display configured to display data to the user; and
a user input interface configured to receive data inputted by the user.

51. The mobile inhaler of claim 43, wherein the processing equipment is configured to collect data comprising at least one of a number of inhalations by a user per day, a duration of inhalations by a user, a time at which an inhalation takes place, and a volume of an inhalation.

52. The mobile inhaler of claim 43, wherein the liquid comprises nicotine, and wherein the processing equipment is configured to collect data comprising intake of the nicotine by the user during inhalation.

53. A mobile inhaler system comprising:
a mobile inhaler configured to atomize a liquid for inhalation by a user, wherein the mobile inhaler comprises:
processing equipment comprising:
memory configured to store information about the user, and
a communcations interface; and
a liquid pump system configured for pumping the liquid; and
a vibrating mesh atomizer configured for atomizing the liquid, wherein the liquid pump system is further configured to pump the liquid to the atomizer;
an external device communicatively coupled to the communications interface by a commuications link.

54. The mobile inhaler system of claim 53, wherein the external device is communicatively coupled to the communications interface using at least one of RFID, Bluetooth protocol, and an Internet Protocol.

55. The mobile inhaler system of claim 53, wherein the external device comprises at least one of a wearable device, a handheld device, a smart phone, and a tablet computer.

56. The mobile inhaler system of claims 53, wherein the processing equipment is configured to determine a dosage of the liquid based at least in part on stored information about the user.

57. The mobile inhaler system of claim 53, wherein the memory is further configured to store information about a plurality of users.

58. The mobile inhaler system of claim 57, wherein the processing equipment is configured to determine a dosage of the liquid based at least in part on stored information about the plurality of users.

59. The mobile inhaler system of any of claims 56 and 58, wherein determining the dosage comprises determining at least one of an amount of the liquid, a duration of atomization, a length of time, and an atomization rate.

60. The mobile inhaler system of claim 53, wherein the processing equipment further comprises a communication interface configured to send and receive data from a network.

61. The mobile inhaler system of claim 60, wherein the processing equipment is configured to transmit data to an external device over the network.

62. The mobile inhaler system of claim 53, wherein the processing equipment further comprises:
a user interface display configured to display data to the user; and
a user input interface configured to receive data inputted by the user.

63. The mobile inhaler system of claim 53, wherein the processing equipment is configured to collect data comprising at least one of a number of inhalations by a user per day, a duration of inhalations by a user, a time at which an inhalation takes place, and a volume of an inhalation.

64. The mobile inhaler system of claim 53, wherein the liquid comprises nicotine, and wherein the processing equipment is configured to collect data comprising intake of the nicotine by the user during inhalation.

65. The mobile inhaler system of claim 64, wherein the external device is configured to measure one or more biomarkers of the user indicative of nicotine exposure.

66. The mobile inhaler system of claim 64, wherein the external device is configured to measure one or more biomarkers of the user indicative of conventional smoking.

67. The mobile inhaler system of any of claims 65 and 66, wherein the external device is configured to communicate to the mobile inhaler a measured level of the one or more biomarkers.

68 The mobile inhaler system of claim 67, wherein the processing equipment is configured to control nicotine delivery to the user based at least in part on the one or more biomarkers.

69. The mobile inhaler system of claim 64, wherein the mobile inhaler further comprises a touchscreen configured to display a visual analog scale user interface, and configured to display prompts the user to self-evaluate the intensity of craving symptoms, wherein the touchscreen is coupled to the processing equipment.

70. The mobile inhaler system of claim 64, wherein the memory is further configured to store information about a plurality of users, and wherein the processing equipment is configured to determine a desired usage pattern of the device based on the stored information about the plurality of users, wherein the usage pattern comprises a nicotine level.

71. The mobile inhaler system of claim 64, wherein the memory is further configured to store information about a plurality of users, the communications interface is configured to transmit the stored information about the plurality of users, and wherein the external device is configured to determine a desired usage pattern of the device based on the stored information about the plurality of users, wherein the usage pattern comprises a nicotine level.

72. The mobile inhaler system of claim 53, wherein at least one of the mobile inhaler and the exeternal device is communicatively coupled to a network, and wherein the mobile inhaler system is configured to provide a measurement indicative of the user's use of the mobile inhaler to a client coupled to the network.

73. The mobile inhaler system of claim 72, wherein the measurement comprises at least one of a nicotine intake of the user and a biomarker level of the user.

74. The mobile inhaler system of claim 53, wherein the mobile inhaler and the external device are communicatively coupled to a client device running an external application.

75. The mobile inhaler system of claim 53, further comprising a capsule configured to store the liquid and comprising identification means configured to store information about the liquid in the capsule wherein at least one of the mobile inhaler and the external device is configured to store historical information about liquids that have been used in the mobile inhaler.

76. A mobile inhaler comprising:
a capsule comprising a liquid and a slidable seal;
a piston pump configured to pump the liquid, the piston pump comprising:
an electric motor comprising a shaft,
a transmission mechanism coupled to the shaft, and
a piston coupled to the transmission mechanism and arranged at one end of the capsule, wherein the piston is configured to apply force to the slidable seal;
a vibrating mesh membrane coupled to the capsule and configured to atomize the liquid; and
control circuitry electrically coupled to the electric motor and the vibrating mesh membrane, and configured to:
control a rotation of the electric motor; and
control an actuation of the vibrating mesh membrane.

77. The mobile inhaler of claim 76, wherein the transmission mechanism comprises a rack and pinion mechanism.

78. The mobile inhaler of claim 77, wherein the pinion is rigidly coupled to the shaft and the rack is rigidly coupled to the piston.

79. The mobile inhaler of claim 76, wherein the transmission mechanism comprises a screw and nut mechanism.

80. The mobile inhaler of claim 79, wherein the screw is rigidly coupled to the shaft and the nut is rigidly coupled to the piston.

81. The mobile inhaler of claim 76, further comprising a mouthpiece configured to be inhaled from by a user.

82. The mobile inhaler of claim 81, wherein the capsule is comprised at least partially within the mouthpiece.

83. The mobile inhaler of claim 82, wherein the capsule is comprised completely within the mouthpiece.

84. The mobile inhaler of claim 76, wherein the electric motor comprises a stepper motor, and wherein the control circuitry comprises a stepper motor controller.

85. The mobile inhaler of claim 76, wherein the electric motor comprises a DC motor, and wherein the control circuitry comprises a DC motor controller.

86. The mobile inhaler of claim 76, wherein the control circuitry is configured to determine a flow rate of the liquid based at least in part on a rotation of the shaft of the electric motor.

87. The mobile inhaler of claim 76, wherein the control circuitry is configured to determine a flow rate of the liquid based at least in part on an atomization rate of the liquid by the vibrating mesh membrane.

88. The mobile inhaler of claim 76, wherein the control circuitry is configured to identify the liquid.

89. The mobile inhaler of claim 88, wherein the capsule comprises an identifier, and wherein the control circuitry is further configured to:
detect the identifier, and
identify the liquid based at least in part on the identifier.

90. The mobile inhaler of claim 88, wherein the control circuitry is further configured to:
receive user input, and
identify the liquid based at least in part on the user input.

91. The mobile inhaler of any of claims 89 and 90, wherein the control circuitry is further configured to determine a desired flow rate of the liquid based at least in part on the identifying the liquid.

92. The mobile inhaler of claim 76, further comprising a membrane holder configured to engage the capsule, wherein:
the capsule comprises a sealing cap;
the vibrating mesh membrane is comprised in the membrane holder;
the membrane holder comprises a membrane chamber adjacent to the vibrating mesh membrane; and
the membrane holder comprises a needle coupled to the membrane chamber and configured to pierce the sealing cap thereby allowing the liquid to flow from the capsule, through the needle, and to the membrane chamber.

93. The mobile inhaler of claim 76, further comprising a power source coupled to the control circuitry.

94. The mobile inhaler of claim 76, further comprising a port configured to transmit at least one of electrical power and data to and from an external device.

95. The mobile inhaler of claim 76, further comprising a pressure sensor electrically coupled to the control circuitry and configured to sense a pressure of the liquid in at least one of the capsule and the membrane chamber.

96. The mobile inhaler of claim 95, wherein the control circuitry is further configured to control at least one of the rotation of the electric motor and the actuation of the vibrating mesh membrane based at least in part on the pressure of the liquid.

97. The mobile inhaler of claim 76, further comprising a pressure sensor electrically coupled to the control circuitry and configured to sense an inhalation of the user by sensing an air pressure in the inhaler.

98. A mobile inhaler comprising:
a capsule holder coupled to a mouthpiece;
a body coupled to the capsule holder;
a capsule arranged in the capsule holder, wherein the capsule comprises a liquid and a slidable seal;
a piston coupled to the slidable seal and a transmission mechanism;
an electric motor coupled to the transmission mechanism an arranged in the body;
a membrane holder arranged in the capsule holder and configured to engage the capsule, wherein the membrane holder comprises a vibrating mesh membrane configured to atomize the liquid; and
control circuitry electrically coupled to the electric motor and the vibrating mesh membrane, configured to:
control a motion of the electric motor; and
control a motion of the vibrating mesh membrane.

99. A liquid management system for a mobile inhaler comprising:
a membrane holder comprising:
a vibrating mesh membrane;
a membrane chamber in contact with the vibrating mesh membrane; and
a needle coupled to the membrane chamber;
a capsule comprising a liquid and a slidable seal, wherein the needle is configured to pierce the capsule, thereby allowing the liquid to flow through the needle to the membrane chamber; and
a piston pump coupled to the slidable seal and configured to apply force to the slidable seal to pump the liquid through the needle to the membrane chamber.

100. The liquid management system of claim 99, wherein the piston pump comprises:
a piston in contact with the slidable seal;
a transmission mechanism coupled to the piston; and
an electric motor coupled to the transmission mechanism.

101. The liquid management system of claim 99, further comprising control circuitry electrically coupled to the piston pump and the vibrating mesh membrane and configured to:
control a flow rate of the piston pump; and
control a flow rate of the vibrating mesh membrane.

102. A control system configured to manage operation of a mobile inhaler, the control system comprising:
a power source;
an electrical port; and
control circuitry coupled to the power source and the electrical port, and configured to:
manage power interactions of the power supply;
manage charging of the power source via the electrical port;
manage data communication with an external device via the electrical port;
identify a liquid to be atomized;
determine at least one operating parameter;
control a piston pump; and
control a vibrating mesh membrane.

103. The control system of claim 102, wherein the operating parameter comprises a flow rate of the liquid.

104. The control system of claim 102, wherein the piston pump comprises an electrical motor and wherein the control circuitry is configured to control a flow rate of the liquid by controlling a motion of the electric motor.

105. The control system of claim 102, wherein the control circuitry is configured to control a flow rate of the liquid by controlling a motion of the vibrating mesh membrane.

106. The control system of claim 102, further comprising a pressure sensor coupled to the control circuitry and configured to sense a pressure of the liquid.

107. The control system of claim 106, wherein the control circuitry is further configured to control at least one of the piston pump and the vibrating mesh membrane based at least in part on the pressure of the liquid.

108. The control system of claim 102, further comprising a pressure sensor coupled to the control circuitry and configured to sense a pressure of air in the mobile inhaler to detect an inhalation.

109. A method of managing operation of a mobile inhaler, the method comprising:
identifying a liquid comprised within a capsule;
determining a desired operating parameter;
detecting an inhalation of a user;
in response to the detecting, controlling a piston pump configured to pump the liquid to a vibrating mesh membrane based at least in part on the operating parameter; and
in response to the detecting, controlling the vibrating mesh membrane to atomize the liquid based at least in part on the operating parameter.

110. The method of claim 109, further comprising selecting an operating mode.

111. The method of claim 110, wherein the operating mode comprises generating aerosol in proportion to an amount of airflow.

112. The method of claim 110, wherein the operating mode comprises generating a known amount of liquid to be atomized.

113. The method of claim 112, wherein an atomization rate held constant.

114. The method of claim 110, wherein the operating mode comprises filling a liquid membrane chamber with liquid, and then atomizing the accumulated liquid until the liquid membrane chamber is empty.

115. The method of claim 110, wherein the operating mode comprises purging liquid from the apparatus, after a capsule is removed.

116. The method of claim 109, further comprising receiving input to a user interface.
